(19) United States
(12) Patent Application Publication
(10) Pub. No.: US 2008/0182777 A1
(43) Pub. Date: Jul. 31, 2008

(54) Regulation of cell migration and adhesion

(76) Inventors: Chandra J. Panchal, London (CA); Jinzi Jason Wu, Dollard-des-Ormeaux (CA); Richard Beliveau, Montreal (CA); Marcia Ruiz, Ste-Genevieve (CA); Seema Garde, Montreal (CA); Borhane Annabi, Brossard (CA); Sylvie Lamy, Montreal (CA); Mounia Bouzeghrane, Montreal (CA); Luc Daigneault, Laval (CA); Robert Hawkins, Cheshire (GB)

Correspondence Address: Kirkpatrick & Lockhart Preston Gates Ellis LLP;(FORMERLY KIRKPATRICK & LOCKHART NICHOLSON GRAHAM), STATE STREET FINANCIAL CENTER, One Lincoln Street, BOSTON, MA, 02111-2950, US

(21) Appl. No.: 11/807,589

(22) Filed: May 29, 2007

Related U.S. Application Data
(63) Continuation of application No. 11/004,270, filed on Dec. 2, 2004, now abandoned.
Application No. 11/004,270 is a continuation-in-part of application No. 10/948,229, filed on Sep. 24, 2004, now abandoned.

(30) Foreign Application Priority Data
Sep. 26, 2003 (CA) 2,441,695

(51) Int. Cl. A61K 38/00

(52) U.S. Cl. 514/2

(57) ABSTRACT

Matrix metalloproteinases (MMPs) play an important role in morphogenesis, angiogenesis, wound healing, and in certain disorders such as rheumatoid arthritis, tumor invasion and metastasis. MMPs are thought to be regulated by a variety of cytokines, growth factors, hormones and phorbol esters. This regulation occurs on three levels; alteration of gene expression, activation of the latent zymogen and inhibition by the tissue inhibitors of metalloproteinases (TIMP). We report here a new agent that regulates the level of MMPs.

This application is a continuation-in-part and claims benefit of priority of U.S. patent application Ser. No. 10/948,229 filed on Sep. 24, 2004 and the benefit of priority of Canadian patent application no. 2,441,695 filed on Sep. 26, 2003, the entire content of which are incorporated herein by reference.

### FIELD OF THE INVENTION

The present invention relates to methods and compositions for treating or preventing cell adhesion, migration, protein secretion. More particularly, the present invention relates to a method for treating or preventing cell adhesion, migration or protein secretion by administration of PSP94 family members and related compounds to a mammal.

### BACKGROUND OF THE INVENTION

Cell adhesion is a process by which cells associate with each other, migrate towards a specific target or localize within the extra-cellular matrix. As such, cell adhesion constitutes one of the fundamental mechanisms underlying numerous biological phenomena. For example, cell adhesion is responsible for the adhesion of hematopoietic cells to endothelial cells and the subsequent migration of those hemopoietic cells out of blood vessels and to the site of injury. As such, cell adhesion plays a role in pathologies such as inflammation and immune reactions in mammals.

Investigations into the molecular basis for cell adhesion have revealed that various cell-surface macromolecules; collectively known as cell adhesion molecules or receptors, mediate cell-cell and cell-matrix interactions. For example, proteins of the superfamily called “integrins” are key mediators in adhesive interactions between hematopoietic cells and their microenvironment (M. E. Hemler, Ann. Rev. Immunol., 8, p. 365 (1990)).

Matrix metalloproteinases (MMPs) play an important role in morphogenesis, angiogenesis, wound healing, and in certain disorders such as rheumatoid arthritis, tumor invasion and metastasis (Birkedal-Hansen, 1995, Curr. Opin. Cell Biol. 7:728-735). MMPs are involved, for example, in physiological function where rearrangements of basement membranes occur.

Five subfamilies of MMPs have been recognized: collagenases, gelatinases, stromelysins, matrilysins, and membrane-type MMPs (MT-MMPs). Most of these enzymes contain propeptide, catalytic and hemopexin domains and are involved in the degradation of collagens, proteoglycans and various glycoproteins. MMPs are secreted as inactive zymogens (pro-MMPs) and their activation seems to be a prerequisite for their function. In vivo activation of pro-MMPs involves the removal of the propeptide by serine proteases (e.g., trypsin, plasmin, etc.). Stimulation or repression of most pro-MMP synthesis is regulated at the transcriptional level by growth factors and cytokines.

Post-translational regulation of MMP activity, on the other hand, is controlled by tissue inhibitors of MMPs (“TIMPs”), four of which have been characterized and designated as TIMP-1, TIMP-2, TIMP-3, and TIMP-4 (Gomez et al., 1997, Eur. J. Cell. Biol. 74:111-122). TIMP-1 is involved in the activation of MMP-9, while TIMP-2 is involved in the activation of MMP-2.

MMP-2 (gelatinase A) and MMP-9 (gelatinase B) hydrolyze basement membrane (extracellular matrix (ECM) protein and non-ECM protein (including collagen)) and have therefore been incriminated in the mechanism of tumor invasion and metastasis. MMP-9 is also involved in inflammation, atheroscelerotic plaque rupture, tissue remodeling, wound healing, mobilization of matrix-bound growth factors, processing of cytokines, pulmonary fibrosis, osteoarthritis (Fujisawa et al., J. Biochem. 125:966, 1999), asthma (Oshita, Y. Thorax 2003; 58:757-760) multiple sclerosis (Opdenakker, G, et al, The Lancet Neurology, 2:747-756, 2000). Its expression correlates, for example, with the desmoplasia (abnormal collagen deposition) that accompanies pancreatic cancer, with the metastasis to lymph nodes by human breast carcinoma cells and with the invasion of regional vessels in giant cell tumors of bones. MMP-9 expression is associated with multiple sclerosis and autoimmune inflammation (e.g., autoimmune encephalomyelitis). MMP-9 may be elevated in gingival crevicular fluid and saliva in patients with gingivitis and periodontal diseases. Determination of MMP-9 activity and/or level has been found useful in the follow-up and in the assessment of prognosis in breast and lung cancer patients (Ranunculo, Int. J. Cancer; Iizasa, Clinical Cancer Research) suggesting a good correlation between MMP-9 with the tumor burden and the clinical status.

MMP-2 plasma levels and activity are elevated in patients with acute myocardial infarction (Ml) and may be involved in post-MI complications. Injury of the vascular wall during coronary interventions (PCI) has been shown to increase MMP-2. The expression of MMP-2 is also increased in the brain of individual with multiple sclerosis.

MT1-MMP (MMP-14) can be activated intracellularly. MT1-MMP contains a motif of basic amino acids upstream of the catalytic domain that are thought to act as endoproteolytic processing signals to furin via the trans-golgi network. MT1-MMP is processed to an activated proteinase through a process involving post-translational endoproteolysis, further processed by furin via the trans-Golgi network and then secreted in an active form. The main mechanism of pro-MMP-2 activation involves the zymogen forming a complex at the cell surface with MT1-MMP and TIMP-2. Cleavage of pro-MMP-2 is also thought to involve binding to integrins. MMP-2 also activates MMP-9.

Evidences show that MMPs are overexpressed in cancer cells. However, in situ hybridization results indicated that stromal fibroblasts found at the proximity of cancer cells as well as vascular cells, inflammatory cells such as macrophages and neutrophils and not only the cancer cells expresses some MMP family members. Thus, there is a significant role of other cells expressing MMP in the contribution to cancer progression.

Failed human hearts examined at autopsy or explanation exhibit alterations of the extracellular matrix (e.g. due to changes in collagen). Modulation of the balance between matrix synthesis and degradation is important in the process of ventricular remodelling and in the pathophysiology of heart failure. Support for the importance of the ECM and activity of matrix metalloproteinases in the development of chronic heart failure has been demonstrated both in animal models of heart diseases and in humans.

Pharmaceutical application of compounds which inhibit the expression of MMPs offers a new approach to cancer treatment as well as treatment for nerve healing, degenerative cartilagenous diseases, decubitus ulcers, arthritis, Alzheimer's disease, wound healing, proliferative retinopathy, proliferative renal diseases, multiple sclerosis, corneal ulcers, uncontrolled tissue remodelling and fertility problems.

Rho GTPase (e.g. RhoA) play a role in several cellular processes, by activating downstream targets that regulates; cell polarization, cell-cell adhesion, cell-matrix adhesion, cell morphology, cell motility, membrane trafficking, cytoskeletal microfilaments reorganization, focal adhesion formation, migration, differentiation, apoptosis, smooth muscle contraction and cell proliferation. Some of these targets lead, for example, to activation of serum response factors.

Rho GTPases have been linked with several neurological processes including neuronal migration and polarization, axon guidance and dendrite formation, as well as synaptic organization and plasticity (Luo L., Nat. Rev. Neuroseci. 1, 173-180, 2000). Rho GTPase as therefore been found associated with neurodegenerative disorders (e.g., X-chromosome linked forms of mental retardation, amyotropic lateral sclerosis) and other diseases, such as, faciogenital dysplasia, Wiskott-Aldrich syndrome, diaphanous (non-syndromic deafness), Tangier disease, etc.

Prostate secretory protein (PSP94) constitutes one of the three predominant proteins found in human seminal fluid along with prostate specific antigen (PSA) and prostatic acid phosphatase (PAP). PSP94 has a molecular weight of 10.7 kDa and contains 10 cysteine residues. The cDNA and the gene coding for PSP94 have been cloned and characterized.

PSP94 inhibits the growth of tumor cells (see U.S. Pat. No. 5,428,011 to Seth et al., the entire content of which is incorporated herein by reference). Tumor growth inhibition by PSP94 fragment such as PCK3145, has also been observed in animal models (see International application No. PCT/CA01/01463 to Garde, S. et al., published under No.: WO02/33090, the entire content of which is incorporated herein by reference). PSP94 also reduces the development of skeletal metastasis (see International application No.: PCT/CA02/01737 to Rabbani, S. et al., published under No.: WO03/039576, the entire content of which is incorporated herein by reference). This latter characteristic was observed by a reduction in calcium levels and hind limb paralysis following administration of PSP94 to animal modeling prostate cancer.

Follicle stimulating hormone seems to be involved in the regulation of some MMPs and TIMPs, at least in Sertoli cells (see for example; Mol. Cell. Endocrinol. 118:37-46, 1996; Biol. Reprod. 62:1040-1046, 2000; Mol. Cell. Endocrinol. 189: 25-35, 2002). In testis, follicle stimulating hormone (FSH) has been shown to induce the expression and secretion of MMP-2, MMP-9, TIMP-1 and TIMP-2 from Sertoli cells in vitro. In addition to its role in normal testicular and ovarian functions, FSH is also involved in stimulation of ovarian, endometrial and prostate tumor cell proliferation and is therefore implicated in tumor progression. PSP94 has been shown to lower FSH levels (Thakur et al., 1981, Ind. J. Exp. Biol. Vol. 19:303-313) and also interfere in the binding of FSH to its receptor using testicular membrane preparations (Vijayalakshmi et al., Int. J. Androl. (1981) 691-702).

### SUMMARY OF THE INVENTION

The present invention relates to regulation of cellular adhesion and/or migration by a PSP94 family member.

The present invention further relates to the regulation of protein secretion from a cell by a PSP94 family member.

This invention also relates to the regulation (either directly or indirectly) of matrix metalloproteinases (MMPs), (e.g., MMP-9, MMP-2, MT1-MMP, etc.) by PSP94 family members. More particularly, the present invention relates to the use of a PSP94 family member for the treatment of a condition related to the activity or expression of MMPs or pro-MMPs and/or to antagonize MMPs or pro-MMPs mediated cellular events (e.g., intracellular transduction mechanisms).

The present invention further provides a mean to control cellular Rho GTPase levels (e.g. RhoA) and activity by contacting a cell with a PSP94 family member. In accordance with the present invention, the PSP94 family member may further be used to control activation of Rho GTPase downstream effectors.

The present invention, in one aspect thereof, provides a method of preventing, inhibiting or suppressing cell adhesion in a mammal which may comprise the step of administering to the mammal a compound (or pharmaceutical composition comprising a compound) selected from the group consisting of a) SEQ ID NO.:5, b) a SEQ ID NO.:5 derivative able to reduce (i.e., reducing) cell adhesion or able to induce (i.e., inducing) shedding of an integrin from the cell surface, c) a SEQ ID NO.:5 fragment able to reduce cell adhesion or able to induce shedding of an integrin from the cell surface, d) a SEQ ID NO.:5 analog able to reduce cell adhesion or able to induce shedding of an integrin from the cell surface, and e) combination of any one of a) through d) thereof or any other PSP94 family member.

In accordance with the present invention the method may be used for preventing, inhibiting or suppressing, for example, cell-adhesion associated inflammation, a cell-adhesion associated immune or autoimmune response, etc. Further in accordance with the present invention, the method may be used to treat or prevent a disease selected from the group consisting of arthritis, psoriasis, transplantation rejection, multiple sclerosis, diabetes, inflammatory bowel disease or any disease for which prevention, inhibition or suppression of cell adhesion is desired or needed.

The present invention further provides in an additional aspect, the use of a compound selected from the group consisting of a) SEQ ID NO.: 5, b) a SEQ ID NO.:5 derivative able to reduce (i.e., reducing) cell adhesion or able to induce (i.e., inducing) shedding of an integrin from the cell surface, c) a SEQ ID NO.:5 fragment able to reduce cell adhesion or able to induce shedding of an integrin from the cell surface, d) a SEQ ID NO.:5 analog able to reduce cell adhesion or able to induce shedding of an integrin from the cell surface, and e) combination of any one of a) through d) thereof or any other PSP94 family member, in the prevention inhibition or suppression of cell adhesion in a mammal.

In accordance with the present invention, the adhesion may be an adhesion mediated though (with the help of) an integrin. Further in accordance with the present invention, the integrin may be, for example, CD44.

The present invention provides, in an additional aspect thereof, a method of preventing, inhibiting or suppressing cell migration in a mammal which may comprise the step of administering to the mammal, a compound (or pharmaceutical composition comprising a compound) selected from the group consisting of a) SEQ ID NO.:5, b) a SEQ ID NO.:5 derivative able to reduce cell migration, c) a SEQ ID NO.:5 fragment able to reduce cell migration, d) a SEQ ID NO.:5 analog able to reduce cell migration, and e) combination of any one of a) through d) thereof or any other PSP94 family member.

The present invention further relates to the use of a compound selected from the group consisting of a) SEQ ID NO.:5, b) a SEQ ID NO.:5 derivative able to reduce (i.e., reducing) cell migration, c) a SEQ ID NO.:5 fragment able to reduce cell migration (for example, in a migration assay as described herein), d) a SEQ ID NO.:5 analog able to reduce cell migration, and e) combination of any one of a) through d) thereof or any other PSP94 family member, in the prevention inhibition or suppression of cell migration in a mammal.

The present invention therefore provides a treatment of a disease for which prevention, inhibition or suppression of cell migration is desired or needed.

In an additional aspect, the present invention relates to a method of inhibiting or lowering protein secretion in a mammal, which may comprise the step of administering to the mammal a compound (a pharmaceutical composition comprising a compound) which may be selected from the group consisting of a) SEQ ID NO.: 5, b) a SEQ ID NO.:5 derivative able to reduce (i.e., reducing) secretion of a protein (for example in a cell base assay described herein), c) a SEQ ID NO.:5 fragment able to reduce secretion of a protein, d) a SEQ ID NO.:5 analog able to reduce secretion of a protein, and e) combination of any one of a) through d) thereof or any other PSP94 family member.

“Secretion of a protein” or “protein secretion” is to be understood herein as the process in which a protein travels from within the intracellular space out to the extra-cellular environment.

In accordance with the present invention, the secretion of a protein may be for example, a constitutive secretion or an induced secretion, etc. Further in accordance with the present invention, the protein may be selected, for example, from the group of gelatinases or from the group consisting of a matrix metalloproteinase and a pro-matrix metalloproteinase. The matrix metalloproteinase may be MMP-2. The pro-matrix metalloproteinase may be pro-MMP-2. The matrix metalloproteinase may also be MMP-9 and the pro-matrix metalloproteinase may be pro-MMP-9.

In yet a further aspect the present invention relates to the use of a compound selected, for example, from the group consisting of a) SEQ ID NO.: 5, b) a SEQ ID NO.:5 derivative able to reduce secretion of a protein, c) a SEQ ID NO.:5 fragment able to reduce secretion of a protein, d) a SEQ ID NO.:5 analog able to reduce secretion of a protein, and e) combination of any one of a) through d) thereof or any other PSP94 family member, in the inhibition or lowering of protein secretion in a mammal.

The present invention therefore provides a treatment of a disease for which inhibition or lowering of protein secretion is desired or needed.

In an additional aspect, the present invention provides a method of inducing RhoGTPase expression in a mammal comprising the step of administering to the mammal a compound which may be selected from the group consisting of a) SEQ ID NO.:5, b) a SEQ ID NO.:5 derivative able to induce RHoA protein, gene or mRNA expression in a cell based assay, c) a SEQ ID NO.:5 fragment able to induce RHoA protein, gene or mRNA expression in a cell based assay, d) a SEQ ID NO.:5 analog able to induce RHoA protein, gene or mRNA expression in a cell based assay, and; e) combination of any one of a) through d) thereof or any other PSP94 family member.

In yet an additional aspect, the present invention relates to the use of a compound selected, for example, from the group consisting of a) SEQ ID NO.: 5, b) a SEQ ID NO.:5 derivative able to induce RHoA protein, gene or mRNA expression in a cell based assay, c) a SEQ ID NO.:5 fragment able to induce RHoA protein, gene or mRNA expression in a cell based assay, d) a SEQ ID NO.:5 analog able to induce RHoA protein, gene or mRNA expression in a cell based assay, and e) combination of any one of a) through d) thereof or any other PSP94 family member, in the induction of RhoGTPase expression in a mammal.

In accordance with the present invention, the RhoGTPase may be, for example, RhoA.

In another aspect the present invention relates to the use of a compound selected, for example, from the group consisting of a) SEQ ID NO.: 5, b) a SEQ ID NO.:5 derivative c) a SEQ ID NO.:5 fragment, d) a SEQ ID NO.:5 analog, and e) combination of any one of a) through d) thereof or any other PSP94 family member in the manufacture of a pharmaceutical composition for inducing RhoGTPase expression in a mammal, for preventing, inhibiting or suppressing cell adhesion in a mammal, for preventing, inhibiting or suppressing cell migration in a mammal or for inhibiting or lowering protein secretion in a mammal.

The present invention therefore provides a treatment of a disease for which induction of RhoGTPase is desired or needed.

In yet another aspect the present invention relates to pharmaceutical compositions comprising a pharmaceutically acceptable carrier and a compound selected, for example, from the group consisting of a) SEQ ID NO.: 5, b) a SEQ ID NO.:5 derivative c) a SEQ ID NO.:5 fragment, d) a SEQ ID NO.:5 analog, and e) combination of any one of a) through d) thereof or any other PSP94 family member for inducing RhoGTPase expression in a mammal, for preventing, inhibiting or suppressing cell adhesion in a mammal, for preventing, inhibiting or suppressing cell migration in a mammal or for inhibiting or lowering protein secretion in a mammal.

In a further aspect the present invention relates to a compound selected, for example, from the group consisting of a) SEQ ID NO.: 5, b) a SEQ ID NO.:5 derivative c) a SEQ ID NO.:5 fragment, d) a SEQ ID NO.:5 analog, and e) combination of any one of a) through d) thereof or any other PSP94 family member for inducing RhoGTPase expression in a mammal, for preventing, inhibiting or suppressing cell adhesion in a mammal, for preventing, inhibiting or suppressing cell migration in a mammal or for inhibiting or lowering protein secretion in a mammal.

In an additional aspect, the present invention relates to a compound member of the PSP94 family for use in the treatment of a condition related to the activity or the expression of a protease (e.g., a serine protease). The condition may happen through the activity or expression of the protease itself or onto another factor (e.g., a factor which may be part of a cascade of event activated by the protease) which may be responsible for the condition.

In another aspect, the present invention provides a compound member of the PSP94 family for use in the treatment of a condition related, for example, to the activity or to the expression of a polypeptide which may be, for example, selected from the group consisting of matrix metalloproteinases and pro-matrix metalloproteinases.

The present invention therefore provides a treatment of a disease for which reduction in the levels or activity of a matrix metalloproteinases or pro-matrix metalloproteinases is desired or needed. More particularly, diseases for which circulating (in the blood, or other bodily fluid) levels of a matrix metalloproteinases or pro-matrix metalloproteinases needs to be reduced are encompassed by the present invention.

PSP94, PSP94 derivatives, PCK3145, PCK3145 derivatives, fragments, analogues and homologues thereof may therefore find utility in cancer treatment, wound healing, anti-angiogesis, anti-inflammation, anti-osteoarthritis, inhibition of hair growth, reduction of degradation of some cytokine (e.g., IFN-beta) as well as for skin treatment (e.g., prevention of blistering photo-aging, psoriasis), wound healing, tissue remodeling, pulmonary fibrosis, etc

It is to be understood herein that a member of the PSP94 family may be selected, for example, from the group consisting of PSP94 (SEQ ID NO.:1), a PSP94 fragment, a PSP94 derivative, a PSP94 analogue, PCK3145 (SEQ ID NO.:5), a PCK3145 fragment, a PCK3145 derivative and a PCK3145 analogue. A PCK3145 derivative may be, for example, as defined in SEQ ID NO.:7. PSP94 family members therefore also include, for example, SEQ ID NO.:2, SEQ ID NO.: 3, SEQ ID NO.:4, SEQ ID NO.:6, as well as SEQ ID NO.: 9 to 98.

More particularly, the member of the PSP94 family (PSP94 family member) may be selected, for example, from the group consisting of;
a) SEQ ID NO.:1,
b) a SEQ ID NO.:1 derivative which may be able to reduce (in a tissue, a cell or cell environment (e.g., extracellular environment)) the activity or the level of expression of a polypeptide selected from the group consisting of a pro-matrix metalloproteinase and a matrix metalloproteinase (e.g., MMP-2 and/or pro-MMP-2),
c) a SEQ ID NO.:1 fragment which may be able to reduce the activity or the level of expression of a polypeptide selected from the group consisting of a pro-matrix metalloproteinase and a matrix metalloproteinase (e.g., MMP-2 and/or pro-MMP-2),
d) SEQ ID NO.:1 analogue which may be able to reduce the activity or the level of expression of a polypeptide selected from the group consisting of a pro-matrix metalloproteinase and a matrix metalloproteinase (e.g., MMP-2 and/or pro-MMP-2),
e) SEQ ID NO.:5,
f) a SEQ ID NO.:5 derivative which may be able to reduce the activity or the level of expression of a polypeptide selected from the group consisting of a pro-matrix metalloproteinase and a matrix metalloproteinase (e.g., MMP-2 and/or pro-MMP-2),
g) a SEQ ID NO.:5 fragment which may be able to reduce the activity or the level of expression of a polypeptide selected from the group consisting of a pro-matrix metalloproteinase and a matrix metalloproteinase (e.g., MMP-2 and/or pro-MMP-2),
h) a SEQ ID NO.:5 analogue which may be able to reduce the activity or the level of expression of a polypeptide selected from the group consisting of a pro-matrix metalloproteinase and a matrix metalloproteinase (e.g., MMP-2 and/or pro-MMP-2),
i) SEQ ID NO.:7, and
j) combination of any one of a) through i) thereof.

In accordance with the present invention the SEQ ID NO.: 1 fragment may be selected, for example, from the group consisting of SEQ ID NO.:4 and SEQ ID NO.:6.

Also in accordance with the present invention the SEQ ID NO.:1 derivative may be selected, for example, from the group consisting of SEQ ID NO.:2 and SEQ ID NO.:3.

In a further aspect, the present invention provides the use of a PSP94 family member for the treatment of a condition related to the expression or related to the (e.g., biological, enzymatic) activity of a polypeptide which may be selected, for example, from the group consisting of matrix metalloproteinases and pro-matrix metalloproteinases.

In yet a further aspect, the present invention relates to the use of a PSP94 family member for the manufacture of a medicament (or pharmaceutical composition) for the treatment of a condition related to the expression or activity of a polypeptide which may be, for example, selected from the group consisting of matrix metalloproteinases and pro-matrix metalloproteinases.

In accordance with the present invention, the condition may be selected from the group consisting of inflammation, atheroscelerotic plaque rupture, skin disease, uncontrolled tissue remodeling and pulmonary fibrosis or any other condition or utility described herein.

In yet another aspect, the present invention relates to the use of a PSP94 family member for reducing or controlling the development or spreading of metastasis or metastatic cancer (i.e, cancer progression to other (secondary) sites or spreading of tumor cells to other sites) other than skeletal metastasis.

In another aspect, the present invention relates to the use of a PSP94 family member for the promotion of wound healing, for reducing (inhibiting) angiogenesis, for reducing (preventing) inflammation, for preventing atheroscelerotic plaque rupture, for skin treatment, for treating osteoarthritis, for treating pulmonary fibrosis or for the inhibition of (unwanted) hair growth.

In a further aspect, the present invention provides a pharmaceutical composition for treating a condition which may be related to the activity and/or to the expression (level) of a polypeptide, which may be, selected from the group consisting of matrix metalloproteinases and pro-matrix metalloproteinases, the pharmaceutical composition may comprise;
a PSP94 family member, and;
a pharmaceutically acceptable carrier.

In yet a further aspect, the present invention provides a method for treating a patient having a condition related to the activity and/or expression of a polypeptide selected from the group consisting of matrix metalloproteinases and pro-matrix metalloproteinases, the method comprising administering to the patient a compound which is a member of the PSP94 family.

In another aspect, the present invention relates to a method of treating a patient having a metastatic cancer or a metastasis other than skeletal metastasis, the method comprising administering to the patient a PSP94 family member.

In an additional aspect, the present invention relates to a matrix metalloproteinase regulation drug and/or a pro-matrix metalloproteinase regulation drug comprising a PSP94 family member.

In another aspect, the present invention provides a compound able to reduce the expression or activity of a polypeptide selected from the group consisting of a matrix metalloproteinases and a pro-matrix metalloproteinases, the compound may comprise or consist essentially of the amino acid sequence identified in SEQ ID NO.:5 and may further comprise a stabilizing group (e.g. a group increasing in vivo stability of the compound or polypeptide without affecting deleteriously the biological activity of the compound or polypeptide) covalently attached to an amino acid of the (SEQ ID NO.:5) sequence.

In accordance with the present invention the group may be, for example, an acetylaminomethyl group attached to a sulfur atom of a cysteine or a polyethylene glycol (PEG) group attached to at least one amino acid of the sequence or any other modification which improves a desired property (e.g., stability) of the compound/polypeptide.

In yet another aspect the present invention relates to a compound selected, for example, from the group consisting of a) SEQ ID NO.: 5, b) a SEQ ID NO.:5 derivative c) a SEQ ID NO.:5 fragment, d) a SEQ ID NO.:5 analog, and e) combination of any one of a) through d) 25 thereof or any other PSP94 family member for controlling (reducing) protein secretion or for reducing the levels of a matrixmetalloproteinase or pro-matrixmetalloproteinase levels in a mammal in need thereof.

In a further aspect, the present invention provides a method for evaluating the efficacy of a treatment with a PSP94 family member in a patient having a metastatic cancer or metastasis, the method may comprise, for example, the steps of
a) collecting a serum sample from the patient after treatment of the patient with a PSP94 family member;
b) measuring the (serum) levels of a polypeptide which may be selected from the group consisting of MMP-9 and pro-MMP-9 in the sample obtained in step a) and;
c) comparing measured levels of step b) with another MMP-9 and/or pro-MMP-9 level selected from the group consisting of levels measured from a normal individual, standard levels or levels measured before treatment of the individual.

The method may also comprise the step of establishing the clinical outcome of the patient based on the comparison of the measured levels.

In accordance with the present invention, the method for evaluating the efficacy of a PSP94 treatment may also measure any other parameters which might correlate with the level of expression of the polypeptide (MMP-9 and/or pro-MMP-9) such as for example, RNA levels.

In accordance with the present invention, the matrix metalloproteinase may be, for example, MMP-2 or may be MMP-9 or any other MMPs. Also in accordance with the present invention, the pro-matrix metalloproteinase may be, for example, pro-MMP-2 or may be pro-MMP-9 or any other pro-MMPs.

A “PSP94 family member” or “a member of the PSP94 family” is to be understood herein as any polypeptide originating from PSP94. For example, “PSP94 family members” may comprise wild type PSP94 (SEQ ID NO.:1) a PSP94 fragment, a PSP94 derivative, a PSP94 analogue, PCK3145 (SEQ ID NO.:5), a PCK3145 fragment, a PCK3145 derivative, a PCK3145 analogue, etc.

A “fragment” is to be understood herein as a polypeptide originating from a portion of an original or parent sequence. Fragments encompass polypeptides having truncations of one or more amino acids, wherein the truncation may originate from the amino terminus (N-terminus), carboxy terminus (C-terminus), or from the interior of the protein. A fragment may comprise the same sequence as the corresponding portion of the original sequence. For example, SEQ ID NO.: 4, SEQ ID NO.: 5 and SEQ ID NO.: 6 fall into the definition of “a PSP94 fragment”; when considering PSP94 (SEQ ID NO.:1) as an original sequence.

A “derivative” is to be understood herein as a polypeptide originating from an original sequence or from a portion of an original sequence and which may comprise one or more modification; for example, one or more modification in the amino acid sequence (e.g., an amino acid addition, deletion, insertion, substitution etc.), one or more modification in the backbone or side-chain of one or more amino acid, or an addition of a group or another molecule to one or more amino acids (side-chains or backbone). For example, SEQ ID NO.: 2, SEQ ID NO.: 3 and SEQ ID NO.: 7 fall into the definition of “a PSP94 derivative”; when considering PSP94 (SEQ ID NO.:1) as an original sequence.

It is to be understood herein that SEQ ID NO.: 7 may fall into the definition of “a PCK3145 derivative” or “SEQ ID NO.:5 derivative) when considering PCK3145 (SEQ ID NO.:5) as an original sequence. The addition of polyethylene glycol group (i.e., pegylation) to PCK3145 (SEQ ID NO.:5 or SEQ ID NO.: 7) also falls within the definition of “a PCK3145 derivative”.

An “analogue” is to be understood herein as a molecule having a biological activity and chemical structure similar to that of a polypeptide described herein. An “analogue” may have sequence similarity with that of an original sequence or a portion of an original sequence and may also have a modification of its structure as discussed herein. For example, an “analogue” may have at least 90% sequence similarity with an original sequence or a portion of an original sequence. An “analogue” may also have, for example; at least 70% or even 50% sequence similarity (or less, i.e., at least 40%) with an original sequence or a portion of an original sequence. Also, an “analogue” may have, for example, 50% sequence similarity to an original sequence with a combination of one or more modification in a backbone or side-chain of an amino acid, or an addition of a group or another molecule, etc.

Thus, biologically active polypeptides in the form of the original polypeptides, fragments (modified or not), analogues (modified or not), derivatives (modified or not), homologues, (modified or not) of PSP94 and PCK3145 are encompassed by the present invention.

Therefore, any polypeptide having a modification compared to an original polypeptide (e.g., PSP94, PCK3145) which does not destroy significantly a desired biological activity is encompassed herein. It is well known in the art, that a number of modifications may be made to the polypeptides of the present invention without deleteriously affecting their biological activity. These modifications may, on the other hand, keep or increase the biological activity of the original polypeptide or may optimize one or more of the particularity (e.g. stability, bioavailability, etc.) of the polypeptides of the present invention which, in some instance might be desirable. Polypeptides of the present invention comprises for example, those containing amino acid sequences modified either by natural processes, such as posttranslational processing, or by chemical modification techniques which are known in the art. Modifications may occur anywhere in a polypeptide including the polypeptide backbone, the amino acid side-chains and the amino- or carboxy-terminus. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslational natural processes or may be made by synthetic methods. Modifications comprise for example, without limitation, pegylation, acetylation, acylation, addition of acetomidomethyl (Acm) group, ADP-ribosylation, alkylation, amidation, biotinylation, carbamoylation, carboxyethylation, esterification, covalent attachment to fiavin, covalent attachment to a heme moiety, covalent attachment of a nucleotide or nucleotide derivative, covalent attachment of drug, covalent attachment of a marker (e.g., fluorescent, radioactive, etc.), covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination, etc. It is to be understood herein that more than one modification to the polypeptides described herein are encompassed by the present invention to the extent that the biological activity is similar to the original (parent) polypeptide.

As discussed above, polypeptide modification may comprise, for example, amino acid insertion (i.e., addition), deletion and substitution (i.e., replacement), either conservative or non-conservative (e.g., D-amino acids, desamino acids) in the polypeptide sequence where such changes do not substantially alter the overall biological activity of the polypeptide which is, for example, to reduce the level of expression of matrix metalloproteinases or pro-matrix metalloproteinases and/or to reduce their enzymatic activity.

Example of substitutions may be those, which are conservative (i.e., wherein a residue is replaced by another of the same general type or group) or when wanted, non-conservative (i.e., wherein a residue is replaced by an amino acid of another type). In addition, a non-naturally occurring amino acid may substitute for a naturally occurring amino acid (i.e., non-naturally occurring conservative amino acid substitution or a non-naturally occurring non-conservative amino acid substitution).

As is understood, naturally occurring amino acids may be sub-classified as acidic, basic, neutral and polar, or neutral and non-polar. Furthermore, three of the encoded amino acids are aromatic. It may be of use that encoded polypeptides differing from the determined polypeptide of the present invention contain substituted codons for amino acids, which are from the same type or group as that of the amino acid be replaced. Thus, in some cases, the basic amino acids Lys, Arg and His may be interchangeable; the acidic amino acids Asp and Glu may be interchangeable; the neutral polar amino acids Ser, Thr, Cys, Gln, and Asn may be interchangeable; the non-polar aliphatic amino acids Gly, Ala, Val, Ile, and Leu are interchangeable but because of size Gly and Ala are more closely related and Val, Ile and Leu are more closely related to each other, and the aromatic amino acids Phe, Trp and Tyr may be interchangeable.

It should be further noted that if the polypeptides are made synthetically, substitutions by amino acids, which are not naturally encoded by DNA (non-naturally occurring or unnatural amino acid) may also be made.

A non-naturally occurring amino acid is to be understood herein as an amino acid which is not naturally produced or found in a mammal. A non-naturally occurring amino acid comprises a D-amino acid, an amino acid having an acetylaminomethyl group attached to a sulfur atom of a cysteine, a pegylated amino acid, etc. The inclusion of a non-naturally occurring amino acid in a defined polypeptide sequence will therefore generate a derivative of the original polypeptide. Non-naturally occurring amino acids (residues) include also the omega amino acids of the formula NH₂(CH₂)ₙCOOH wherein n is 2-6, neutral nonpolar amino acids, such as sarcosine, t-butyl alanine, t-butyl glycine, N-methyl isoleucine, norleucine, etc. Phenylglycine may substitute for Trp, Tyr or Phe; citrulline and methionine sulfoxide are neutral nonpolar, cysteic acid is acidic, and ornithine is basic. Proline may be substituted with hydroxyproline and retain the conformation conferring properties.

It is known in the art that analogues may be generated by substitutional mutagenesis and retain the biological activity of the polypeptides of the present invention. These analogues have at least one amino acid residue in the protein molecule removed and a different residue inserted in its place. For example, one site of interest for substitutional mutagenesis may include but are not restricted to sites identified as the active site(s), or immunological site(s). Other sites of interest may be those, for example, in which particular residues obtained from various species are identical. These positions may be important for biological activity. Examples of substitutions identified as “conservative substitutions” are shown in table 1. If such substitutions result in a change not desired, then other type of substitutions, denominated “exemplary substitutions” in table 1, or as further described herein in reference to amino acid classes, are introduced and the products screened.

In some cases it may be of interest to modify the biological activity of a polypeptide by amino acid substitution, insertion, or deletion. For example, modification of a polypeptide may result in an increase in the polypeptide's biological activity, may modulate its toxicity, may result in changes in bioavailability or in stability, or may modulate its immunological activity or immunological identity. Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (Ile)
(2) neutral hydrophilic: Cysteine (Cys), Serine (Ser), Threonine (Thr)
(3) acidic: Aspartic acid (Asp), Glutamic acid (Glu)
(4) basic: Asparagine (Asn), Glutamine (Gln), Histidine (His), Lysine (Lys), Arginine (Arg)
(5) residues that influence chain orientation: Glycine (Gly), Proline (Pro); and aromatic: Tryptophan (Trp), Tyrosine (Tyr), Phenylalanine (Phe)

Non-conservative substitutions will entail exchanging a member of one of these classes for another.

TABLE 1
amino acid substitution

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

Example of biologically active analogues of PCK3145 (SEQ ID NO: 5) exemplified by amino acid substitutions is illustrated below.

| | |
|---|---|
| Position | 1 5 10 15 |
| PCK3145 | E W Q T D N C E T C T C Y E T |
| (SEQ ID | X₁ W Q X₂ D X₁ C X₁ X₂ C X₂ C X₃ X₁ X₂ |
| NO.:88) | |

For example, X₁ may be glutamic acid (i.e., glutamate) (Glu), aspartic acid (aspartate) (Asp), or asparagine (Asn), X₂ may be threonine (Thr) or serine (Ser) and X₃ may be tyrosine (Tyr) or phenylalanine (Phe). Any replacement of an original residue in SEQ ID NO.:5 with a conserved amino acid (i.e. conservative substitution) is encompassed by the present invention.

Another example of a PCK3145 (SEQ ID NO: 5) analogue may include, for example, a polypeptide as exemplified in SEQ ID NO.:88 or any other polypeptide having at least one conservative amino acid substitution (illustrated in bold below) as defined in Table 1, such as, for example;

(SEQ ID NO.:92)

Glu Tyr Gln Thr Asp Asn Cys Glu Thr Cys Thr Cys
Tyr Glu Thr

(SEQ ID NO.:93)

Glu Trp Asn Thr Asp Asn Cys Glu Thr Cys Thr Cys
Tyr Glu Thr

(SEQ ID NO.:94)

Glu Trp Gln Thr Asp Gln Ser Glu Thr Cys Thr Cys
Tyr Asp Thr

Examples of a PCK3145 (SEQ ID NO: 5) derivative may include, for example, a polypeptide having an addition in one or both of the terminal region (amino-terminal or carboxy-terminal) as illustrated in SEQ IDs No.: 9 to 87, a peptide having a stabilizing group such as exemplified in SEQ ID NO.:7, or a peptide having one or more repeats of SEQ ID No.:5 such as exemplified in SEQ ID NOs.: 89 to 91, a polypeptide having at least one D-amino acid as exemplified in SEQ ID No. 98 and combination thereof.

An example of a PCK3145 (SEQ ID NO: 5) fragment may include, for example, a polypeptide having a truncation in one or both of the amino acid terminal region as illustrated below.

(SEQ ID NO.:95)

Trp Gln Thr Asp Asn Cys Glu Thr Cys Thr Cys Tyr
Glu Thr

(SEQ ID NO.:96)

Gln Thr Asp Asn Cys Glu Thr Cys Thr Cys Tyr Glu
Thr

(SEQ ID NO.:97)

Glu Trp Gln Thr Asp Asn Cys Glu Thr Cys Thr Cys

Polypeptides may be either naturally occurring (that is to say, substantially purified or isolated from a natural source) or synthetic (for example, by performing site-directed mutagenesis on the encoding DNA or made by other synthetic methods such as chemical synthesis). It is thus apparent that the polypeptides of the invention can be either naturally occurring or recombinant (that is to say prepared from the recombinant DNA techniques) or made by chemical synthesis (e.g., organic synthesis).

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

The formulations include those suitable for oral, rectal, ophthalmic, (including intravitreal or intracameral) nasal, topical (including buccal and sublingual), vaginal or parenteral (including subcutaneous, intramuscular, intravenous, intradermal, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into associate the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. An example of a topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tamports, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Further, as used herein “pharmaceutically acceptable carrier” or “pharmaceutical carrier” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's orfixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

It is to be understood herein, that if a “range” or “group” of substances (e.g. amino acids), substituents” or the like is mentioned or if other types of a particular characteristic (e.g. temperature, pressure, chemical structure, time, etc.) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,
with respect to a temperature greater than 100° C., this is to be understood as specifically incorporating herein each and every individual temperature state, as well as sub-range, above 100° C., such as for example 101° C., 105° C. and up, 110° C. and up, 115° C. and up, 110 to 135° C., 115° C. to 135° C., 102° C. to 150° C., up to 210° C., etc.;
   and similarly with respect to other parameters such as, concentrations, elements, etc . . . .

It is in particular to be understood herein that the polypeptides of the present invention each include each and every individual polypeptide described thereby as well as each and every possible mutant, variant, homolog, analogue or else whether such mutant, variant, homolog, analogue or else is defined as positively including particular polypeptides, as excluding particular polypeptides or a combination thereof; for example an exclusionary definition for a polypeptide analogue (e.g. X₁WQX₂DX₁CX₁X₂CX₂CX₃X₁X₂ (SEQ ID NO:88)) may read as follows: “provided that when one of X₁ is glutamic acid and X₂ is threonine X₃ may not be phenylalanine”. Another type of exclusionary definition may read “other than skeletal metastasis” or “provided that said condition is not skeletal metastasis”.

It is also to be understood herein that “g” or “gm” is a reference to the gram weight unit; that “C” is a reference to the Celsius temperature unit.

### BRIEF DESCRIPTION OF DRAWINGS

In drawings which illustrates exemplary embodiment of the present invention;

FIG. 1 is a picture of a zymography gel showing the effect of the PCK3145 derivative (SEQ ID NO.:7) on MMP-9 levels and activity on collagen type 1-treated MatLyLu cells (first lane:marker; second lane:cells; third lane:cells and collagen; fourth lane:cells, collagen and 500 μg/ml of SEQ ID NO.:7; fifth lane:cells, collagen and 1 mg/ml of SEQ ID NO.:7),

FIG. 2 is a picture of a western blot showing the effect of the PCK3145 derivative (SEQ ID NO.:7) on MMP-9 expression level (first lane: MMP9 standard; second lane:cells; third lane:cells and collagen; fourth lane:cells, collagen and 100 μg/ml of SEQ ID NO.:7; fifth lane: cells, collagen and 500 μg/ml of SEQ ID NO.:7; sixth lane: cells, collagen and 1 mg/ml of SEQ ID NO.:7),

FIG. 3 is a picture of a zymography gel showing the effect of the PCK3145 derivative (SEQ ID NO.:7) on MMP-2 levels and VEGF-induced MMP-2 levels and a histogram expressing the results in a quantitative manner (PD=PD98059, PCK=PCK3145 derivative),

FIG. 4A is a histogram quantifying U-87 cell migration on hyaluronic acid (HA) in the presence or absence of the PCK3145 derivative (Ctl=control, PCK=PCK3145 derivative),

FIG. 4B is a histogram quantifying U-87 cell adhesion to hyaluronic acid (HA) in the presence or absence of the PCK3145 derivative (Ctl=control, PCK=PCK3145 derivative),

FIG. 5 is a picture of a western blot showing the effect of the PCK3145 derivative on MT1-MMP cell expression and CD44 shedding from the cell surface,

FIG. 6A is a picture of a western blot showing the effect of the PCK3145 derivative on MT1-MMP expression in transfected cells and RhoA expression in cells and histograms expressing the results in a quantitative manner,

FIG. 6B is a picture of a gel illustrating the effect of the PCK3145 derivative on Rho RNA levels, and;

FIG. 7 is a histogram illustrating the effect of the PCK3145 derivative on the constitutive expression of SEAP.

### DETAILED DESCRIPTION OF THE INVENTION

Polypeptides which are members of the PSP94 family include; wild type PSP94 as defined in SEQ ID NO.: 1, a recombinant PSP94 as defined in SEQ ID NO.:2 and PSP94 derivatives, fragments and analogues as defined, for example in the amino acid SEQ ID NO.: 3, SEQ ID NO.:4, SEQ ID NO.:5, SEQ ID NO.:6 and SEQ ID NO.:7.

PCK3145 (SEQ ID NO.:5) was chosen as a representative of the PSP94 family based on previous encouraging results of tumor growth inhibition observed in animals.

Test compound. The wild type amino acid sequence of PCK3145 has been disclosed, for example, in international application No.: PCT/CA01/01463 and is defined herein in SEQ ID NO.: 5. A PCK3145 derivative has been generated by attaching an acetylaminomethyl group to the sulfur atom of each of the three cysteines of PCK3145. These groups stabilize the compound by preventing formation of peptide dimers or polymer by blocking the sulfhydryl group of cysteines. This PCK3145 derivative is defined in SEQ ID NO.: 7. The drug was manufactured by Multiple Peptide Systems (3550) (General Atomics Court, San Diego, Calif.) using standard solid-phase peptide chemistry and lyophilized into a powder. Other type of synthesis or manufacture method may however be performed to make a peptide or polypeptide of the invention. Other PCK3145 derivatives, analogs and fragments (e.g., SEQ IDs NO: 88, 98, etc.) may be generated similarly.

The reconstituted drug used in the present example is made from a solution containing a 20 mg/mL of PCK3145 derivative (SEQ ID NO.:5 derivative); SEQ ID NO.: 7, in a phosphate buffer at pH 7.4 for dilution in sterile saline (0.9% NaCl, BP) prior to intravenous administration. The solutions is filled into Type 1 glass vials, stoppered with Teflon®-faced butyl stoppers, and sealed with flip-off seals.

The drug was administered to patients characterized as having metastatic adenocarcinoma of the prostate, stage 1V prostatic cancer and as having a metastatic hormone resistant prostatic cancer.

Biological samples were drawn during different time points. Plasma samples were placed on dry ice and stored frozen (approximately −70° C.) and subsequently analyzed for total MMP-9 levels.

MMP-9 assay methodology. An Elisa assay measuring total MMP-9, i.e., human active and pro-MMP-9, (Quantikine®, Cat. No.: DMP900, R&D Systems Inc.) was performed on plasma-heparin samples. Plasma samples have been collected from individuals at day 1 (before treatment) and at day 27 of each treatment cycle.

The Quantikine® MMP-9 immunoassay is a solid phase ELISA designed to measure total MMP-9 (92 kDa pro- and 82 kDa active forms) in serum, plasma, saliva, urine and cell culture supernatants. It is calibrated with CHO-cells expressed recombinant human pro-MMP-9 and the antibodies were raised against the recombinant factor. Both antibodies also recognize recombinant human active MMP-9. Natural human MMP-9 showed dose-response curves that were parallel to the standard curves obtained using the recombinant Quantikine® kit standards, indicating that the Quantikine® kit may be used to determine relative mass values of natural human MMP-9.

The assay employs the quantitative sandwich enzyme immunoassay technique. A monoclonal antibody specific for MMP-9 has been pre-coated onto a microplate. Standards and samples are added into the wells, and MMP-9 is thus bound by the immobilized antibody. After washing away unbound substances, an enzyme-linked polyclonal antibody specific for MMP-9 is added to the wells. Following a wash to remove unbound antibody-enzyme reagent, a substrate solution is added to the wells and color develops in proportion to the amount of total MMP-9 (pro and/or active) bound in the initial step. The color development is stopped and the intensity of the color is measured.

Zymography. Zymography is a technique generally used to analyze the activity of matrix metalloproteinases (MMPs) in biological samples. It involves the electrophoretic separation of proteins under denaturing (Sodium Dodecyl Sulfate (SDS)) but non-reducing conditions through a polyacrylamide gel containing gelatin (for example, 10% gel containing 1 mg/ml gelatin for MMP-9 and MMP-2 assays). The resolved proteins are re-natured by exchanging SDS with a non-ionic detergent such as Triton X-100 and the gel is incubated in an incubation buffer for activation of MMP-2 and MMP-9 (for example at 37° C. for 18 hrs). The gel is stained with Coomassie blue and the MMP-2 and MMP-9 bands may be visualized as clear bands against a blue background (i.e., the MMPs degrade the gelatin and are visualized as clear bands; pro MMP-2 is 68 kDa and pro-MMP-9 is 92 kDa). These bands can be quantified using densitometry.

Materials. Cell culture media were obtained from Life Technologies (Burlington, Ontario, Canada) and serum was purchased from Hyclone Laboratories (Logan, Utah). Electrophoresis reagents were purchased from Bio-Rad (Mississauga, Ontario, Canada). The polyclonal (C-1158) and monoclonal (A3) antibodies, used for precipitation and detection, respectively, of VEGFR-2, and the anti-PDGFR pAb (958) were obtained from Santa Cruz Biotechnologies (Santa Cruz, Calif.). Antiphosphotyrosine mAb PY99 was also purchased from Santa Cruz Biotechnologies. Anti-phospho-ERK polyclonal antibodies were from Cell Signaling Technology (Beverly, Mass.). Anti-mouse and anti-rabbit horseradish peroxidase-linked secondary antibodies were purchased from Jackson ImmunoResearch Laboratories (West Grove, Pa.) and enhanced chemiluminescence (ECL) reagents were from Amersham Pharmacia Biotech (Baie d'Urfé, Quebec, Canada). Human recombinant PDGF was obtained from R&D Systems (Minneapolis, Minn.). Micro bicinchoninic acid protein assay reagents were from Pierce (Rockford, Ill.). All other reagents were from Sigma-Aldrich Canada.

Cell culture. Human umbilical vein endothelial cells (HUVEC) and pulmonary aortic smooth muscle cells (PASMC) were obtained from Clonetics and maintained in endothelial cell basal medium-2 (EBM-2; Clonetics) and smooth muscle medium-2 (SmGM-2; Clonetics), respectively. Cells were cultured at 37° C. under a humidified atmosphere containing 5% CO2. For experimental purposes, cells were plated in 8 100-mm plastic dishes at 5,000 cells/cm² and were grown to confluence before overnight serum starvation. Cells were treated with vehicle or with PCK3145 diluted in 0.1 N NaOH, and stimulated with 50 ng/ml VEGF, PDGF or with 1 μM S1P.

Immunoprecipitation and immunoblotting procedures. After treatment, cells were washed once with phosphate-buffered saline (PBS) containing 1 mM sodium orthovanadate and were incubated in the same medium for 1 h at 4° C. The cells were solubilized on ice in lysis buffer (150 mM NaCl, 10 mM Tris-HCl, pH 7.4, 1 mM EDTA, 1 mM EGTA, 0.5% Nonidet P-40, 1% Triton X-100) containing 1 mM sodium orthovanadate. The cells were then scraped from the culture dishes and the resulting lysates were clarified by centrifugation at 10,000 g for 10 min. Protein concentrations were determined using the micro bicinchoninic acid method. For immunoprecipitation studies, lysates were clarified by a 1 h incubation at 4° C. with a mixture of Protein A/Protein G Sepharose beads. After removal of the Sepharose beads by low-speed centrifugation, identical amounts of protein (200 μg) from each sample were transferred to fresh tubes and incubated in lysis buffer overnight at 4° C. in the presence of 2 μg/ml of specific antibodies. Immunocomplexes were collected by incubating the mixture with 25 μl (50% suspension) of Protein A—(rabbit primary antibody) or Protein G—(mouse primary antibody) Sepharose beads, for 2 h. Nonspecifically-bound material was removed by washing the beads three times in 1 ml of lysis buffer containing 1 mM sodium orthovanadate, and bound material was solubilized in 25 μl of two-fold concentrated Laemmli sample buffer, boiled 5 min, and resolved by SDS-PAGE. The proteins were transferred onto polyvinylidene difluoride (PVDF) membranes, blocked 1 h at room temperature with Tris-buffered saline/Tween 20 (147 mM NaCl, 20 mM Tris/HCl, pH 7.5, and 0.1% Tween 20) containing 2% bovine serum albumin and incubated overnight at 4° C. with primary antibody. Immunoreactive bands were revealed after a 1 h incubation with horseradish peroxidase-conjugated anti-mouse or anti-rabbit antibodies, and the signals were visualized by enhanced chemiluminescence (Amersham Biosciences, Baie d'Urfée, QC).

Migration Assays. Transwells filters (8-μm pore size; Costar, Cambridge, Mass.) were pre-coated with 0.5% gelatin/PBS for 24 h at 4° C. The transwells were then washed with PBS and assembled in 24-well plates. The upper chamber of each transwell was filled with 100 μl of HUVEC (1×10⁶ cells/ml) and cells were allowed to adhere for 1 h. Cells were then treated for 2 h by adding 100 μl of 2-fold concentrated drug solution prepared in serum-free medium into the upper chamber and 600 μl of the drug solution into the lower chamber. Migration was initiated by adding VEGF (10 ng/ml), or S1P (1 μM) to the lower chamber. The plate was placed at 37° C. in 5% CO₂/95% air for 4 h. Cells that had migrated to the lower surface of the filters were fixed with 10% formalin phosphate and stained with 0.1% Crystal Violet/20% (v/v) methanol. The migration was quantified using computer-assisted imaging and data are expressed as the average density of migrated cells per four fields (magnification ×50).

Biologically active PSP94 family member. Fragments, derivatives and analogues may be prepared by techniques known in the art (recombinant technology, solid phase synthesis, etc.). The biological activity of derivatives, fragments and analogues may be determined by any of the techniques described herein or known in the field to be relevant for any of the biological activity described above.

The biological activity of a desired polypeptide may be determined, for example, by contacting a cell expressing a metalloproteinase (e.g., MMP-9, MMP-2) and/or pro-metalloproteinase (e.g., pro-MMP-9, pro-MMP-2) with a polypeptide of the present invention (a PSS94 family member (e.g.: original polypeptide, fragment, derivative, analogue, and/or any modified form of an original polypeptide, fragment, derivative or analogue) and, following incubation of the polypeptide and cell, evaluating the levels (inside the cell or in the extracellular environment (supernatant or blood (plasma or serum))) of expression of the metalloproteinase by western blot or the enzymatic activity of the metalloproteinase by zymography as described herein or by any other techniques known in the art to be representative of metalloproteinase activity or expression (e.g., northern blot, PCR, immunochemistry methods, etc.). A modification (e.g., reduction or in some cases an increase) of the level of expression or enzymatic activity of a metalloproteinase (and/or pro-metalloproteinase) will identify a biologically active polypeptide.

The biological activity of a desired polypeptide may also be determined using migration assays. U-87 cells are treated with a polypeptide of the present invention (e.g., any PCK3145 derivative, fragment, analog, such as for example, any one of or combinations of SEQ ID NOs.: 9 to 98). The treated cells are trypsinised, counted, and seeded on HA-coated filters inserted in modified Boyden Chambers as described herein or in the art. Cell migration is allowed to proceed for 2 hours at 37° C. Filters are then stained for cells that have migrated through the filter. A decreased basal U-87 cell migration observed in cells treated with a polypeptide of the present invention is indicative of a biologically active polypeptide (i.e., a biologically active PCK3145 derivative, fragment, analog).

Each putative derivative, fragment or analogue may be tested using this technique or any other similar techniques described herein or known in the art.

### EXAMPLE 1

### In Vivo MMPs Measurements

### MMP-9 Assay Results

Results of MMP-9 levels in patient's plasma, before and after one or more treatment cycle with PCK3145 derivative; SEQ ID NO.: 7 are illustrated in Table 2.

Normal values of healthy volunteers were not determined in this study but lizasa et al., has determined that the normal range of plasma MMP-9 concentrations is about 11.4 to 59.4 ng/ml. Based on theses values, patients were sub-divided into two categories; those having normal value of MMP-9 (below 100 μg/L) and those having an elevated level of MMP-9 (higher than 100 μg/L) at baseline (see column identified as D1C1 in Table 2).

In the normal value MMP-9 category (patients identified as E, F, G, H and I), there was no significant decrease in MMP-9 levels after one cycle of treatment (column identified D27C1) compared to baseline levels. For patients E and G, no decrease in MMP-9 levels was observed compared to baseline values even after 2 cycles of treatment (column identified D27C2). There was still no MMP-9 decrease even after 3 cycles of treatment for patient E (D27C3)

In the elevated MMP-9 category (patients identified as A, B, C and D), a significant decrease was observed for each patient after only one cycle of treatment (see column identified as D27C1). For example a decrease of up to 89% in MMP-9 levels was observed for patient A compared to baseline levels. For patient B, the decrease in MMP-9 was 41% after cycle 1. For patients C and D the decrease at cycle 1 was 90% and 34% respectively.

This decrease was maintained for patients B and C who have received more treatment cycles (see columns identified as D27C2, D27C3 and D27C4). For example, at treatment cycle 2, patient B showed a reduction of 64% of its baseline level of MMP-9. A similar reduction was also measured for patient B at treatment cycle 3; i.e., a 65% reduction, and at treatment cycle 4; a 75% reduction. In the case of patient C, a reduction of 76% in MMP-9 levels was measured at cycle 2.

TABLE 2

| | | | | | | |
|---|---|---|---|---|---|---|
| Pa- | | | | | | Maximum |
| tient | D1C1 | D27C1 | D27C2 | D27C3 | D27C4 | Reduction |

Elevated MMP-9: Baseline = 100 μg/L

| | | | | | | |
|---|---|---|---|---|---|---|
| A | 424 | 47.3 | N.A. | N.A. | N.A. | 89% |
| B | 156.5 | 91.6 | 55.6 | 54.5 | 39.4 | 75% |
| C | 155 | 14.9 | 37.7 | N.A. | N.A. | 90% |
| D | 130.2 | 85.2 | N.A. | N.A. | | 34% |

Normal MMP-9: Baseline = 100 μg/L

| | | | | | |
|---|---|---|---|---|---|
| E | 57.5 | 58 | 60.1 | 101.8 | N.A. |
| F | 53.2 | 73.1 | N.A. | N.A. | N.A. |
| G | 33.9 | 45.4 | 189.6 | | N.A. |
| H | 57.0 | 44.0 | 65 | | |
| I | 22.1 | 18.8 | | | |

N.A. = not applicable

### EXAMPLE 2

### Effect on MMP-9 Secretion

In order to support in vivo results described in Example 1, zymography assays and western blots were performed on cell lines incubated with the PCK3145 derivative (SEQ ID NO.:7).

Therefore, in the present experiment, 2.5×10⁵ MatLyLu tumor cells (American Type Culture Collection No.: JHU-5)) were seeded in T-25 flasks containing RPMI with 10% fetal bovine serum (FBS). After overnight incubation, the cells were washed once with serum free medium and treated with various concentrations of the PCK3145 derivative (500 μg/ml and 1 mg/ml) in the presence of 50 ug/ml collagen type-I in serum free RPMI for 72 hrs. Control cells received 50 ug/ml collagen or only serum free medium.

The media were collected after 72 hours of exposure to the PCK3145 derivative and subjected to gelatin zymography. Zymography for MMP-2 and MMP-9 was performed in SDS-polyacrylamide gel electrophoresis (SDS-PAGE) (10%) containing 0.1% gelatin (Invitrogen). Twenty-four microliters of culture media was mixed with non-reducing sample buffer and subjected to electrophoresis without boiling. After electrophoresis, gels were soaked for 30 minutes in 2.5% Triton X-100 solution with 2-3 washing steps. The gels were then incubated for 18 hours at 37° C. in buffer containing 50 mM Tris/HCl, pH 7.6, 50 mM NaCl, 10 mM CaCl₂ and 0.05% Brij-35. After incubation, the gels were stained with 0.2% Coomassie blue and de-stained until clear proteolytic bands appeared. Gels were scanned with Microtek flatbed scanner (Scanmaker 5 software; Microtek lab, Redondo Beach, Calif.). The band intensities were determined using the Image Quant software (version 5.0) from molecular Dynamics.

The MMP-9 and MMP-2 gelatinase zymography standard were purchased from Chemicon (catalogue no. CC073). One nanogram of purified human pro-MMP-2 and pro-MMP-9 standards were used in every gel run.

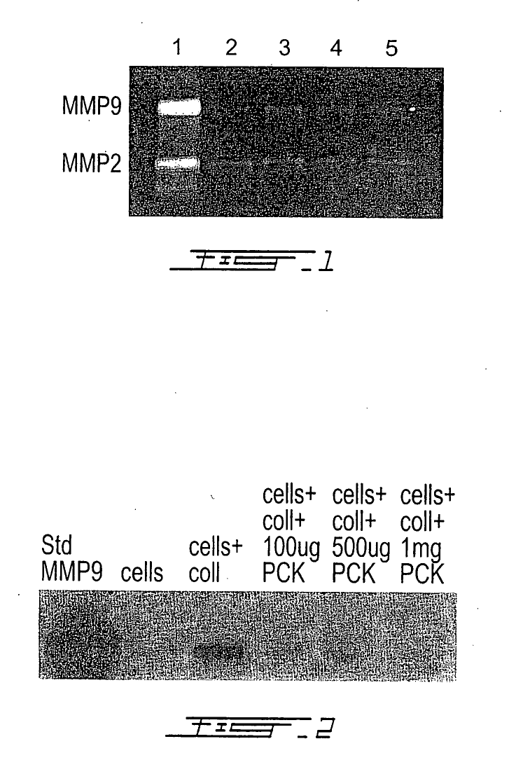

Results of this experiment are illustrated in FIG. 1 and indicate that PCK3145 treatment of MatLyLu cells resulted in a dose-dependent reduction of MMP-9 secreted in the cell culture media, as detected by zymography.

### Western Blot

A western blot experiment was performed in a separate experiment in which MatLyLu cells were treated with 100 ug/ml, 500 ug/ml and 1 mg/ml of the PCK3145 derivative for 72 hrs. At the end of the experiment, the media were collected and concentrated 5 times using Amicon centrifugal filter devices (3500 molecular weight cut-off).

Twenty five microliters samples were separated on SDS-PAGE gel under reducing conditions using pre-cast gels of 4-12% Bis-Tris (Invitrogen). Following electrophoresis, the proteins were transferred on nitrocellulose membrane. Non-specific binding sites were blocked using 5% skimmed milk in 10 mM phosphate buffer saline (PBS) containing 0.05% Tween-20 for 1 hour at room temperature. The membrane was later incubated with a primary antibody (monoclonal, RDI-MMP-9abm-2A5) at a concentration of 1 ug/ml (in 10 mM PBS, containing 0.5% bovine serum albumin (BSA) and 0.05% Tween-20) for 3 hours at room temperature.

The membranes were washed three times in PBS (5 minutes each wash) to remove non-specific binding and they were incubated with the secondary antibody (Rabbit anti-mouse IgG horseradish peroxidase-conjugated (Dako no. 0260)) at a dilution of 1:5000 for one hour. Detection of specific MMP-9 protein was made by incubating the membrane in ECL™ reagent (electro-chemoluminescence, Roche) and exposing to the X-ray film.

Results of this experiment are illustrated in FIG. 2 and again indicate that treatment of MatLyLu cells PCK3145 derivative resulted in a dose-dependent reduction of MMP-9 levels.

### EXAMPLE 3

### Effect on VEGF Induced MMP-2 Secretion

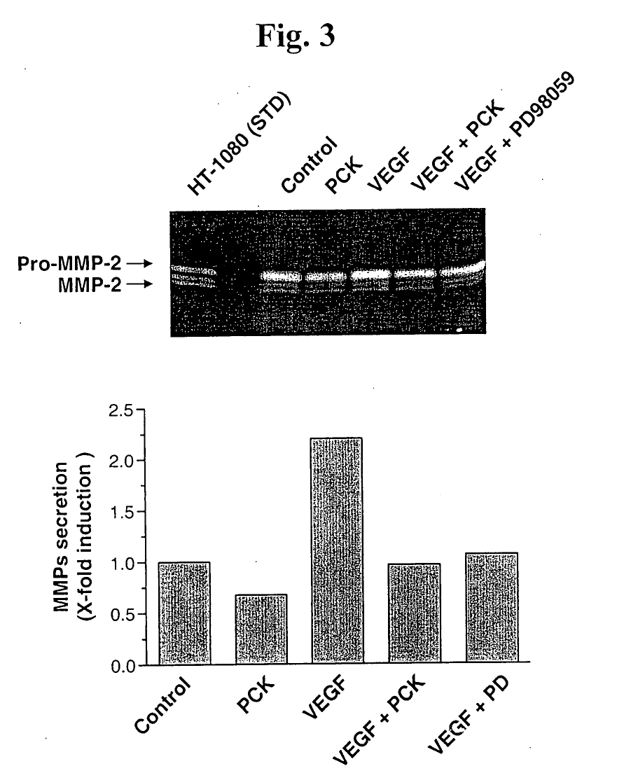

The effect of the PCK3145 derivative (SEQ ID NO.:7) on MMP extracellular levels was assessed by gelatin-zymography in the conditioned media of serum-starved HUVEC. After 16 hours of starvation, HUVEC were stimulated with VEGF in the presence or not of the PCK3145 derivative. A further 24 hours treatment shows that PCK3145 derivative effectively downregulated by approximately 35% the basal proMMP-2 levels in the extracellular media (FIG. 3). The effect of the PCK3145 derivative (300 pg/ml) was also observed on VEGF-induced proMMP-2 secretion as the inhibition was of approximately 50%. When these experiments were performed in serum-free media, but in the presence of the MAPK inhibitor PD98059, VEGF-induced proMMP-2 extracellular levels were also significantly decreased. These results suggest that the effect of the PCK3145 derivative towards MMP secretion is indeed regulated through a MAPK pathway in endothelial cells.

### EXAMPLE 4

### Effect on U-87 Cell Migration on Hyaluronic Acid

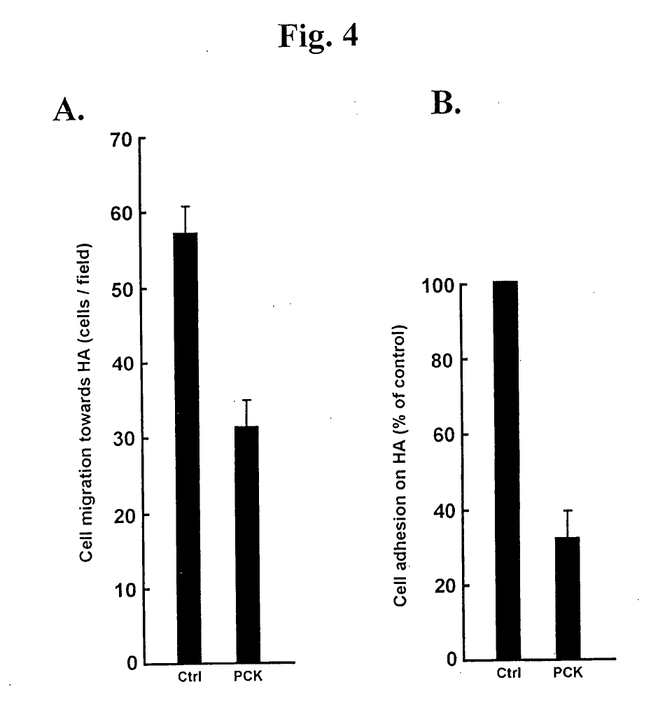

Migration/invasion of cancer cells is a key event in tumor metastasis. In vitro, this process can be reconstituted by plating cells onto ECM-coated filters inserted in modified Boyden chemotactic chambers. The effect of the PCK3145 derivative can be monitored by the number of cells that had migrated comparatively to untreated control cells. In light of previous observations, the diminished migration onto hyaluronic acid (HA) matrice was confirmed. U-87 cells were treated with the PCK3145 derivative (300 ug/ml, 48 hrs), trypsinised, counted, and seeded on HA-coated filters inserted in modified Boyden Chambers. Cell migration was allowed to proceed for 2 hours at 37° C. Filters were then stained for cells that have migrated through the filter. The results show that pretreatment with the PCK3145 derivative decreased basal U-87 cell migration by approximately 3-fold (FIG. 4A). This result was performed for 3 more times with new cell preparations.

### EXAMPLE 5

### Effect on U-87 Cell Adhesion to Hyaluronic Acid (HA)

ECM recognition is a crucial event in the cell adhesion processes involved in tumor progression. This process is mediated and regulated through specialized cell surface receptors or integrins. While recent evidence suggests that a potential crosstalk between soluble MMP and cell surface integrins may regulate the cell's ability to recognize and adhere to its ECM environment, the PCK3145 derivative was tested in its ability to downregulate U-87 cell adhesion onto HA. U-87 cells were treated with the PCK3145 derivative (300 ug/ml, 48 hrs), trypsinised, counted, and seeded on wells coated with 10 ug/ml BSA (bovine serum albumin) or HA. Cells were allowed to adhere for 3 hours. Three independent experiments were performed. Results of these experiments show that adhesion of cells treated with the PCK3145 derivative was significantly diminished on HA by 45-76% (FIG. 4B). Collectively, the inhibitory action of the PCK3145 derivative on ECM recognition and cell adhesion processes suggests that the expression of specific integrins or HA cell surface receptors such as those from the CD44 family could be targeted. Alternatively, such result also suggests that intracellular signalling regulating the activation states of cell surface integrins may be triggered by the PCK3145 derivative. One such potential intracellular protein is the GTPase RhoA, which is likely to mediate mechanisms regulating cytoskeletal morphogenesis.

### EXAMPLE 6

### Effect on CD44 Cell Surface Shedding

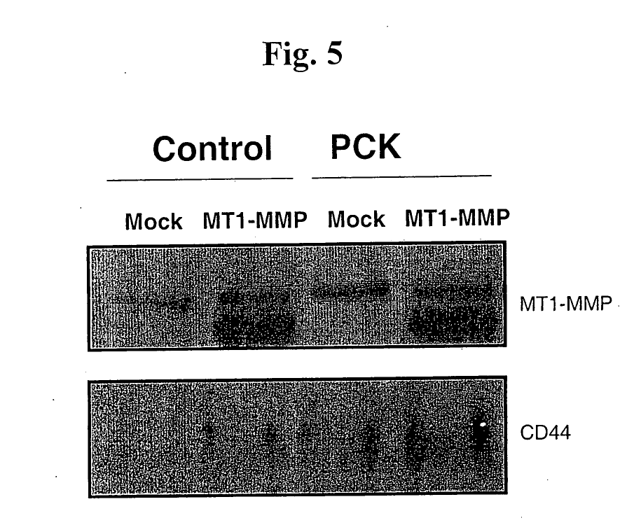

Decreased cell migration and adhesion on HA was observed when U-87 cells were pretreated with the PCK3145 derivative. This can be interpreted as either a potential downregulation of CD44 expression at the cell surface or by a potential cell surface shedding. The latter hypothesis was tested by incubating serum-starve U-87 cells for 24 hours with the PCK3145 derivative (300 ug/ml), a concentration known to antagonize MMP secretion. The conditioned media was then TCA-precipitated and Western blotting an immunodetection for a 75 kDa immunoreactive protein using the anti-CD44 antibody was performed. An increased CD44 cell surface shedding was demonstrated by the strong immunoreactive band observed in the cells which had been pre-treated with PCK3145 derivative (FIG. 5). This effect is also shown in parallel with MT1-MMP-transfected cells. Such effect has been already reported by many groups and is established as one of the MT1-MMP-mediated functions in the regulation of the ECM adhesion. Interestingly, a slight increase in MT1-MMP expression in the cells treated with the PCK3145 derivative was observed that may partially explain how PCK may lead to CD44 shedding. This induction has subsequently been reproduced below. Altogether, these observations provide a rational for the diminished cell migration/adhesion to HA. Moreover, it is tempting to further suggest that this may also be a secondary regulation by the PCK3145 derivative of diminished cell surface docking of MMP-9 to CD44.

### EXAMPLE 7

### Effect on MT1-MMP and RhoA Expression

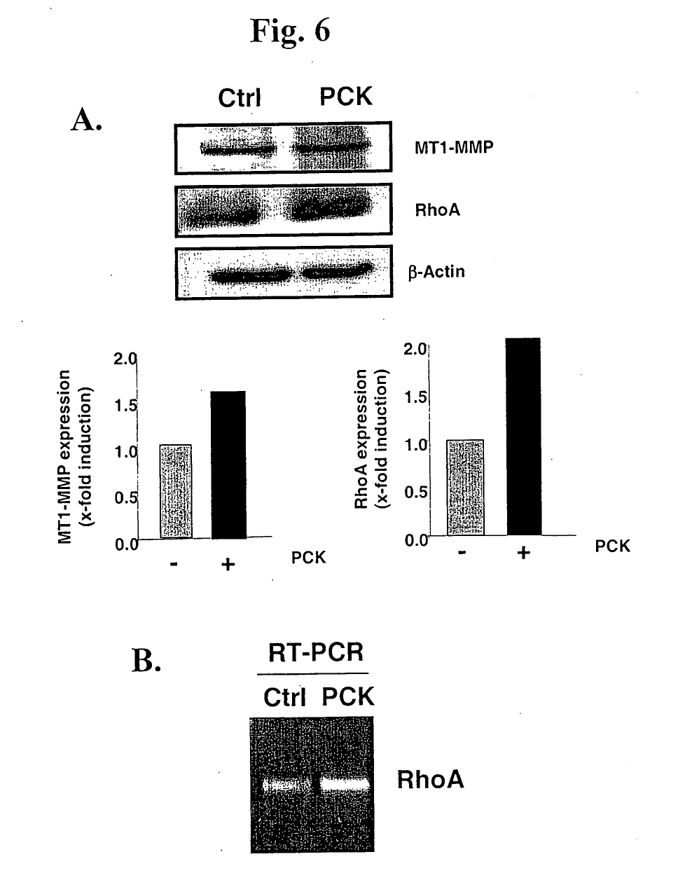

Specific manipulation of the GTPase Rho activity can be used to suppress or enhance the organizational behaviour of endothelial cells as well as it can restrict cancer cells proliferation. In particular, RhoA mediates cell contractility by organizing actin filaments which consequently regulates cell migration. Moreover, recent evidence suggested that RhoA/CD44/MMP-9 colocalized at common cell surface microdomains. Tests were carried out in order to determine whether the PCK3145 derivative affected RhoA gene and protein expression. U-87 cells were either treated with the PCK3145 derivative (300 ug/ml, 48 hrs). Results of this experiment confirm that the PCK3145 derivative induced endogenous RhoA protein expression in U-87 cells as assessed by Western blotting (FIG. 6A). Finally, results show that RhoA protein expression induced by the PCK3145 derivative paralleled that of its gene expression as assessed by reverse transcription-polymerase chain reaction (RT-PCR) (FIG. 6B). Altogether, these results highlight the potential role of RhoA as being an intracellular mediator in the subsequent inhibitory activities of the PCK3145 derivative.

### EXAMPLE 8

### Effect on Constitutive Secretion

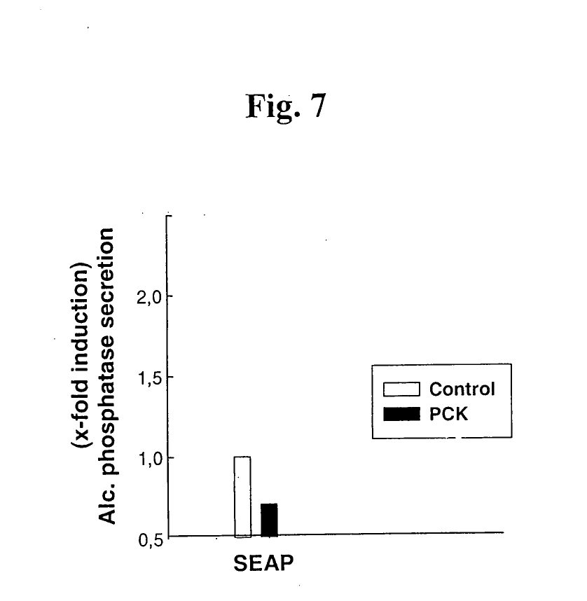

In order to investigate the potential intracellular pathways triggered by the PCK3145 derivative, a gene-reporter assay using the SEAP (Secreted Alkaline Phosphatase) Mercury Profiling Kit (CLONTECH) was performed. This assay enables monitoring of the transcription factors that are triggered by a particular experimental condition by assaying the alkaline phosphatase activity in the extracellular media. In this assay, secretion of the constitutively expressed SEAP was found to be inhibited suggesting a potential effect of the PCK3145 derivative on a more general constitutive secretion pathway (FIG. 7).

The effect of PSP94 family members on MMP-9 and MMP-2 makes them useful for reduction of cancer spreading and invasion of any type of cancer and not only for reduction of skeletal metastasis as disclosed and claimed in International application No.: PCT/CA02/01737.

The content of each publication, patent and patent application mentioned in the present application is incorporated herein by reference.

Although the present invention has been described in details herein and illustrated in the accompanying drawings, it is to be understood that the invention is not limited to the embodiments described herein and that various changes and modifications may be effected without departing from the scope or spirit of the present invention.

| SEQUENCE DESCRIPTION: SEQ ID NO:1: | |
|---|---|
| Ser Cys Tyr Phe Ile Pro Asn Glu Gly Val Pro Gly Asp Ser Thr Arg | |
| 5 10 15 | |
| Lys Cys Met Asp Leu Lys Gly Asn Lys His Pro Ile Asn Ser Glu Trp | |
| 20 25 30 | |
| Gln Thr Asp Asn Cys Glu Thr Cys Thr Cys Tyr Glu Thr Glu Ile Ser | |
| 35 40 45 | |
| Cys Cys Thr Leu Val Ser Thr Pro Val Gly Tyr Asp Lys Asp Asn Cys | |
| 50 55 60 | |
| Gln Arg Ile Phe Lys Lys Glu Asp Cys Lys Tyr Ile Val Val Glu Lys | |
| 65 70 75 80 | |
| Lys Asp Pro Lys Lys Thr Cys Ser Val Ser Glu Trp Ile Ile | |
| 85 90 | |
| SEQUENCE DESCRIPTION: SEQ ID NO:2: | |
| Glu Ala Glu Ala Tyr Val Glu Phe Ser Cys Tyr Phe Ile Pro Asn Glu | |
| 1 5 10 15 | |
| Gly Val Pro Gly Asp Ser Thr Arg Lys Cys Met Asp Leu Lys Gly Asn | |
| 20 25 30 | |
| Lys His Pro Ile Asn Ser Glu Trp Gln Thr Asp Asn Cys Glu Thr Cys | |
| 35 40 45 | |
| Thr Cys Tyr Glu Thr Glu Ile Ser Cys Cys Thr Leu Val Ser Thr Pro | |
| 50 55 60 | |
| Val Gly Tyr Asp Lys Asp Asn Cys Gln Arg Ile Phe Lys Lys Glu Asp | |
| 65 70 75 80 | |
| Cys Lys Tyr Ile Val Val Glu Lys Lys Asp Pro Lys Lys Thr Cys Ser | |
| 85 90 95 | |
| Val Ser Glu Trp Ile Ile | |
| 100 | |
| SEQUENCE DESCRIPTION: SEQ ID NO:3: | |
| Tyr Thr Cys Ser Val Ser Glu Pro Gly Ile | |
| 1 5 10 | |
| SEQUENCE DESCRIPTION: SEQ ID NO:4: | |
| Asn Glu Gly Val Pro Gly Asp Ser Thr Arg Lys Cys Met Asp Leu | |
| 1 5 10 15 | |
| SEQUENCE DESCRIPTION: SEQ ID NO:5: | |
| Glu Trp Gln Thr Asp Asn Cys Glu Thr Cys Thr Cys Tyr Glu Thr | |
| 1 5 10 15 | |
| SEQUENCE DESCRIPTION: SEQ ID NO:6: | |
| Ile Val Val Glu Lys Lys Asp Pro Lys Lys Thr Cys Ser Val Ser Glu | |
| 1 5 10 15 | |
| Trp Ile Ile | |
| SEQUENCE DESCRIPTION: SEQ ID NO:7 (an acetylaminomethyl group | |
| may be attached to the sulfur atom of cysteine 7, of cysteine | |
| 10 and/or of cysteine 12) | |
| Glu Trp Gln Thr Asp Asn Cys Glu Thr Cys Thr Cys Tyr Glu Thr | |
| 1 5 10 15 | |
| SEQUENCE DESCRIPTION: SEQ ID NO:8: | |
| TCATGCTATT TCATACCTAA TGAGGGAGTT CCAGGAGATT CAACCAGGAA ATGCATGGAT | 60 |
| CTCAAAGGAA ACAAACACCC AATAAACTCG GAGTGGCAGA CTGACAACTG TGAGACATGC | 120 |
| ACTTGCTACG AAACAGAAAT TTCATGTTGC ACCCTTGTTT CTACACCTGT GGGTTATGAC | 180 |
| AAAGACAACT GCCAAAGAAT CTTCAAGAAG GAGGACTGCA AGTATATCGT GGTGGAGAAG | 240 |
| AAGGACCCAA AAAAGACCTG TTCTGTCAGT GAATGGATAA TCTAA | 285 |
| SEQUENCE DESCRIPTION: SEQ ID NO:9: | |
| Glu Trp Gln Thr Asp Asn Cys Glu Thr Cys Thr Cys Tyr Glu Thr Glu | |
| 1 5 10 15 | |
| SEQUENCE DESCRIPTION: SEQ ID NO:10: | |
| Glu Trp Gln Thr Asp Asn Cys Glu Thr Cys Thr Cys Tyr Glu Thr Glu | |
| 1 5 10 15 | |
| Ile | |
| SEQUENCE DESCRIPTION: SEQ ID NO:11: | |
| Glu Trp Gln Thr Asp Asn Cys Glu Thr Cys Thr Cys Tyr Glu Thr Glu | |
| 1 5 10 15 | |
| Ile Ser | |
| SEQUENCE DESCRIPTION: SEQ ID NO:12: | |
| Glu Trp Gln Thr Asp Asn Cys Glu Thr Cys Thr Cys Tyr Glu Thr Glu | |
| 1 5 10 15 | |
| Ile Ser Cys | |
| SEQUENCE DESCRIPTION: SEQ ID NO:13: | |
| Glu Trp Gln Thr Asp Asn Cys Glu Thr Cys Thr Cys Tyr Glu Thr Glu | |
| 1 5 10 15 | |
| Ile Ser Cys Cys | |
| 20 | |
| SEQUENCE DESCRIPTION: SEQ ID NO:14: | |
| Glu Trp Gln Thr Asp Asn Cys Glu Thr Cys Thr Cys Tyr Glu Thr Glu | |
| 1 5 10 15 | |
| Ile Ser Cys Cys Thr | |
| 20 | |
| SEQUENCE DESCRIPTION: SEQ ID NO:15: | |
| Glu Trp Gln Thr Asp Asn Cys Glu Thr Cys Thr Cys Tyr Glu Thr Glu | |
| 1 5 10 15 | |
| Ile Ser Cys Cys Thr Leu | |
| 20 | |
| SEQUENCE DESCRIPTION: SEQ ID NO:16: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val | |
| 20 | |
| SEQUENCE DESCRIPTION: SEQ ID NO:17: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser | |
| 20 | |
| SEQUENCE DESCRIPTION: SEQ ID NO:18: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr | |
| 20 25 | |
| SEQUENCE DESCRIPTION: SEQ ID NO:19: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro | |
| 20 25 | |
| SEQUENCE DESCRIPTION: SEQ ID NO:20: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val | |
| 20 25 | |
| SEQUENCE DESCRIPTION: SEQ ID NO:21: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly | |
| 20 25 | |
| SEQUENCE DESCRIPTION: SEQ ID NO:22: | |
| Glu Trp Gln Thr Asp Asn Cys Glu Thr Cys Thr Cys Tyr Glu Thr Glu | |
| 1 5 10 15 | |
| Ile Ser Cys Cys Thr Leu VaL Ser Thr Pro Val Gly Tyr | |
| 20 25 | |
| SEQUENCE DESCRIPTION: SEQ ID NO:23: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp | |
| 20 25 30 | |
| SEQUENCE DESCRIPTION: SEQ ID NO:24: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys | |
| 20 25 30 | |
| SEQUENCE DESCRIPTION: SEQ ID NO:25: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| SEQUENCE DESCRIPTION: SEQ ID NO:26: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn | |
| SEQUENCE DESCRIPTION: SEQ ID NO:27: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys | |
| SEQUENCE DESCRIPTION: SEQ ID NO:28: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln | |
| 35 | |
| SEQUENCE DESCRIPTION: SEQ ID NO:29: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg | |
| 35 | |
| SEQUENCE DESCRIPTION: SEQ ID NO:30: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile | |
| 35 | |
| SEQUENCE DESCRIPTION: SEQ ID NO:31: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe | |
| 35 | |
| SEQUENCE DESCRIPTION: SEQ ID NO:32: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys | |
| 35 | |
| SEQUENCE DESCRIPTION: SEQ ID NO:33: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys | |
| 35 40 | |
| SEQUENCE DESCRIPTION: SEQ ID NO:34: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu | |
| 35 40 | |
| SEQUENCE DESCRIPTION: SEQ ID NO:35: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp | |
| 35 40 | |
| SEQUENCE DESCRIPTION: SEQ ID NO:36: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys | |
| 35 40 | |
| SEQUENCE DESCRIPTION: SEQ ID NO:37: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys | |
| 35 40 | |
| SEQUENCE DESCRIPTION: SEQ ID NO:38: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr | |
| 35 40 45 | |
| SEQUENCE DESCRIPTION: SEQ ID NO:39: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile | |
| 35 40 45 | |
| SEQUENCE DESCRIPTION: SEQ ID NO:40: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val | |
| 35 40 45 | |
| SEQUENCE DESCRIPTION: SEQ ID NO:41: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| SEQUENCE DESCRIPTION: SEQ ID NO:42: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu | |
| SEQUENCE DESCRIPTION: SEQ ID NO:43: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys | |
| 50 | |
| SEQUENCE DESCRIPTION: SEQ ID NO:44: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys | |
| 50 | |
| SEQUENCE DESCRIPTION: SEQ ID NO:45: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp | |
| 50 | |
| SEQUENCE DESCRIPTION: SEQ ID NO:46: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro | |
| 50 | |
| SEQUENCE DESCRIPTION: SEQ ID NO:47: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys | |
| 50 | |
| SEQUENCE DESCRIPTION: SEQ ID NO:48: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys | |
| 50 55 | |
| SEQUENCE DESCRIPTION: SEQ ID NO:49: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr | |
| 50 55 | |
| SEQUENCE DESCRIPTION: SEQ ID NO:50: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr Cys | |
| 50 55 | |
| SEQUENCE DESCRIPTION: SEQ ID NO:51: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr Cys Ser | |
| 50 55 | |
| SEQUENCE DESCRIPTION: SEQ ID NO:52: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr Cys Ser Val | |
| 50 55 | |
| SEQUENCE DESCRIPTION: SEQ ID NO:53: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr Cys Ser Val Ser | |
| 50 55 60 | |
| SEQUENCE DESCRIPTION: SEQ ID NO:54: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr Cys Ser Val Ser Glu | |
| 50 55 60 | |
| SEQUENCE DESCRIPTION: SEQ ID NO:55: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr Cys Ser Val Ser Glu Trp | |
| 50 55 60 | |
| SEQUENCE DESCRIPTION: SEQ ID NO:56: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr Cys Ser Val Ser Glu Trp Ile | |
| 50 55 60 | |
| SEQUENCE DESCRIPTION: SEQ ID NO:57: | |
| Glu Trp Gln Thr Asp Asn Cys Glu Thr Cys Thr Cys Tyr Glu Thr Glu | |
| 1 5 10 15 | |
| Ile Ser Cys Cys Thr Leu Val Ser Thr Pro Val Gly Tyr Asp Lys Asp | |
| 20 25 30 | |
| Asn Cys Gln Arg Ile Phe Lys Lys Glu Asp Cys Lys Tyr Ile Val Val | |
| 35 40 45 | |
| Glu Lys Lys Asp Pro Lys Lys Thr Cys Ser Val Ser Glu Trp Ile Ile | |
| 50 55 60 | |
| SEQUENCE DESCRIPTION: SEQ ID NO:58: | |
| Ser Glu Trp Gln Thr Asp Asn Cys Glu Thr Cys Thr Cys Tyr Glu Thr | |
| 1 5 10 15 | |
| SEQUENCE DESCRIPTION: SEQ ID NO:59: | |
| Asn Ser Glu Trp Gln Thr Asp Asn Cys Glu Thr Cys Thr Cys Tyr Glu | |
| 1 5 10 15 | |
| Thr | |
| SEQUENCE DESCRIPTION: SEQ ID NO:60: | |
| Ile Asn Ser Glu Trp Gln Thr Asp Asn Cys Glu Thr Cys Thr Cys Tyr | |
| 1 5 10 15 | |
| Glu Thr | |
| SEQUENCE DESCRIPTION: SEQ ID NO:61: | |
| Pro Ile Asn Ser Glu Trp Gln Thr Asp Asn Cys Glu Thr Cys Thr Cys | |
| 1 5 10 15 | |
| Tyr Glu Thr | |
| SEQUENCE DESCRIPTION: SEQ ID NO:62: | |
| His Pro Ile Asn Ser Glu Trp Gln Thr Asp Asn Cys Glu Thr Cys Thr | |
| 1 5 10 15 | |
| Cys Tyr Glu Thr | |
| 20 | |
| SEQUENCE DESCRIPTION: SEQ ID NO:63: | |
| Lys His Pro Ile Asn Ser Glu Trp Gln Thr Asp Asn Cys Glu Thr Cys | |
| 1 5 10 15 | |
| Thr Cys Tyr Glu Thr | |
| 20 | |
| SEQUENCE DESCRIPTION: SEQ ID NO:64: | |
| Asn Lys His Pro Ile Asn Ser Glu Trp Gln Thr Asp Asn Cys Glu Thr | |
| 1 5 10 15 | |
| Cys Thr Cys Tyr Glu Thr | |
| 20 | |
| SEQUENCE DESCRIPTION: SEQ ID NO:65: | |
| Gly Asn Lys His Pro Ile Asn Ser Glu Trp Gln Thr Asp Asn Cys Glu | |
| 1 5 10 15 | |
| Thr Cys Thr Cys Tyr Glu Thr | |
| 20 | |
| SEQUENCE DESCRIPTION: SEQ ID NO:66: | |
| Lys Gly Asn Lys His Pro Ile Asn Ser Glu Trp Gln Thr Asp Asn Cys | |
| 1 5 10 15 | |
| Glu Thr Cys Thr Cys Tyr Glu Thr | |
| 20 | |
| SEQUENCE DESCRIPTION: SEQ ID NO:67: | |
| Leu Lys Gly Asn Lys His Pro Ile Asn Ser Glu Trp Gln Thr Asp Asn | |
| 1 5 10 15 | |
| Cys Glu Thr Cys Thr Cys Tyr Glu Thr | |
| 20 25 | |
| SEQUENCE DESCRIPTION: SEQ ID NO:68: | |
| Asp Leu Lys Gly Asn Lys His Pro Ile Asn Ser Glu Trp Gln Thr Asp | |
| 1 5 10 15 | |
| Asn Cys Glu Thr Cys Thr Cys Tyr Glu Thr | |
| 20 25 | |
| SEQUENCE DESCRIPTION: SEQ ID NO:69: | |
| Met Asp Leu Lys Gly Asn Lys His Pro Ile Asn Ser Glu Trp Gln Thr | |
| 1 5 10 15 | |
| Asp Asn Cys Glu Thr Cys Thr Cys Tyr Glu Thr | |
| 20 25 | |
| SEQUENCE DESCRIPTION: SEQ ID NO:70: | |
| Cys Met Asp Leu Lys Gly Asn Lys His Pro Ile Asn Ser Glu Trp Gln | |
| 1 5 10 15 | |
| Thr Asp Asn Cys Glu Thr Cys Thr Cys Tyr Glu Thr | |
| 20 25 | |
| SEQUENCE DESCRIPTION: SEQ ID NO:71: | |
| Lys Cys Met Asp Leu Lys Gly Asn Lys His Pro Ile Asn Ser Glu Trp | |
| 1 5 10 15 | |
| Gln Thr Asp Asn Cys Glu Thr Cys Thr Cys Tyr Glu Thr | |
| 20 25 | |
| SEQUENCE DESCRIPTION: SEQ ID NO:72: | |
| Arg Lys Cys Met Asp Leu Lys Gly Asn Lys His Pro Ile Asn Ser Glu | |
| 1 5 10 15 | |
| Trp Gln Thr Asp Asn Cys Glu Thr Cys Thr Cys Tyr Glu Thr | |
| 20 25 30 | |
| SEQUENCE DESCRIPTION: SEQ ID NO:73: | |
| Thr Arg Lys Cys Met Asp Leu Lys Gly Asn Lys His Pro Ile Asn Ser | |
| 1 5 10 15 | |
| Glu Trp Gln Thr Asp Asn Cys Glu Thr Cys Thr Cys Tyr Glu Thr | |
| 20 25 30 | |
| SEQUENCE DESCRIPTION: SEQ ID NO:74: | |
| Ser Thr Arg Lys Cys Met Asp Leu Lys Gly Asn Lys His Pro Ile Asn | |
| 1 5 10 15 | |
| Ser Glu Trp Gln Thr Asp Asn Cys Glu Thr Cys Thr Cys Tyr Glu Thr | |
| 20 25 30 | |
| SEQUENCE DESCRIPTION: SEQ ID NO:75: | |
| Asp Ser Thr Arg Lys Cys Met Asp Leu Lys Gly Asn Lys His Pro Ile | |
| 1 5 10 15 | |
| Asn Ser Glu Trp Gln Thr Asp Asn Cys Glu Thr Cys Thr Cys Tyr Glu | |
| 20 25 30 | |
| Thr | |
| SEQUENCE DESCRIPTION: SEQ ID NO:76: | |
| Gly Asp Ser Thr Arg Lys Cys Met Asp Leu Lys Gly Asn Lys His Pro | |
| 1 5 10 15 | |
| Ile Asn Ser Glu Trp Gln Thr Asp Asn Cys Glu Thr Cys Thr Cys Tyr | |
| 20 25 30 | |
| Glu Thr | |
| SEQUENCE DESCRIPTION: SEQ ID NO:77: | |
| Pro Gly Asp Ser Thr Arg Lys Cys Met Asp Leu Lys Gly Asn Lys His | |
| 1 5 10 15 | |
| Pro Ile Asn Ser Glu Trp Gln Thr Asp Asn Cys Glu Thr Cys Thr Cys | |
| 20 25 30 | |
| Tyr Glu Thr | |
| 35 | |
| SEQUENCE DESCRIPTION: SEQ ID NO:78: | |
| Val Pro Gly Asp Ser Thr Arg Lys Cys Met Asp Leu Lys Gly Asn Lys | |
| 1 5 10 15 | |
| His Pro Ile Asn Ser Glu Trp Gln Thr Asp Asn Cys Glu Thr Cys Thr | |
| 20 25 30 | |
| Cys Tyr Glu Thr | |
| 35 | |
| SEQUENCE DESCRIPTION: SEQ ID NO:79: | |
| Gly Val Pro Gly Asp Ser Thr Arg Lys Cys Met Asp Leu Lys Gly Asn | |
| 1 5 10 15 | |
| Lys His Pro Ile Asn Ser Glu Trp Gln Thr Asp Asn Cys Glu Thr Cys | |
| 20 25 30 | |
| Thr Cys Tyr Glu Thr | |
| 35 | |
| SEQUENCE DESCRIPTION: SEQ ID NO:80: | |
| Glu Gly Val Pro Gly Asp Ser Thr Arg Lys Cys Met Asp Leu Lys Gly | |
| 1 5 10 15 | |
| Asn Lys His Pro Ile Asn Ser Glu Trp Gln Thr Asp Asn Cys Glu Thr | |
| 20 25 30 | |
| Cys Thr Cys Tyr Glu Thr | |
| 35 | |
| SEQUENCE DESCRIPTION: SEQ ID NO:81: | |
| Asn Glu Gly Val Pro Gly Asp Ser Thr Arg Lys Cys Met Asp Leu Lys | |
| 1 5 10 15 | |
| Gly Asn Lys His Pro Ile Asn Ser Glu Trp Gln Thr Asp Asn Cys Glu | |
| 20 25 30 | |
| Thr Cys Thr Cys Tyr Glu Thr | |
| 35 | |
| SEQUENCE DESCRIPTION: SEQ ID NO:82: | |
| Pro Asn Glu Gly Val Pro Gly Asp Ser Thr Arg Lys Cys Met Asp Leu | |
| 1 5 10 15 | |
| Lys Gly Asn Lys His Pro Ile Asn Ser Glu Trp Gln Thr Asp Asn Cys | |
| 20 25 30 | |
| Glu Thr Cys Thr Cys Tyr Glu Thr | |
| 35 40 | |
| SEQUENCE DESCRIPTION: SEQ ID NO:83: | |
| Ile Pro Asn Glu Gly Val Pro Gly Asp Ser Thr Arg Lys Cys Met Asp | |
| 1 5 10 15 | |
| Leu Lys Gly Asn Lys His Pro Ile Asn Ser Glu Trp Gln Thr Asp Asn | |
| 20 25 30 | |
| Cys Glu Thr Cys Thr Cys Tyr Glu Thr | |
| 35 40 | |
| SEQUENCE DESCRIPTION: SEQ ID NO:84: | |
| Phe Ile Pro Asn Glu Gly Val Pro Gly Asp Ser Thr Arg Lys Cys Met | |
| 1 5 10 15 | |
| Asp Leu Lys Gly Asn Lys His Pro Ile Asn Ser Glu Trp Gln Thr Asp | |
| 20 25 30 | |
| Asn Cys Glu Thr Cys Thr Cys Tyr Glu Thr | |
| 35 40 | |
| SEQUENCE DESCRIPTION: SEQ ID NO:85: | |
| Tyr Phe Ile Pro Asn Glu Gly Val Pro Gly Asp Ser Thr Arg Lys Cys | |
| 1 5 10 15 | |
| Met Asp Leu Lys Gly Asn Lys His Pro Ile Asn Ser Glu Trp Gln Thr | |
| 20 25 30 | |
| Asp Asn Cys Glu Thr Cys Thr Cys Tyr Glu Thr | |
| 35 40 | |
| SEQUENCE DESCRIPTION: SEQ ID NO:86: | |
| Cys Tyr Phe Ile Pro Asn Glu Gly Val Pro Gly Asp Ser Thr Arg Lys | |
| 1 5 10 15 | |
| Cys Met Asp Leu Lys Gly Asn Lys His Pro Ile Asn Ser Glu Trp Gln | |
| 20 25 30 | |
| Thr Asp Asn Cys Glu Thr Cys Thr Cys Tyr Glu Thr | |
| 35 40 | |
| SEQUENCE DESCRIPTION: SEQ ID NO:87: | |
| Ser Cys Tyr Phe Ile Pro Asn Glu Gly Val Pro Gly Asp Ser Thr Arg | |
| 1 5 10 15 | |
| Lys Cys Met Asp Leu Lys Gly Asn Lys His Pro Ile Asn Ser Glu Trp | |
| 20 25 30 | |
| Gln Thr Asp Asn Cys Glu Thr Cys Thr Cys Tyr Glu Thr | |
| 35 40 45 | |
| 2) INFORMATION FOR SEQ ID NO:88: | |

| | |
|---|---|
| (i) | SEQUENCE CHARACTERISTICS: |
| | (A) LENGTH: 15 |
| | (B) TYPE: AMINO ACID |
| | (C) STRANDEDNESS: SINGLE |
| | (D) TOPOLOGY: LINEAR |
| (ii) | MOLECULE TYPE: |
| (ix) | FEATURE: |
| | NAME/KEY: Modified site |
| | LOCATION: 1 |
| | OTHER INFORMATION: The residue in |
| | this position is either glutamic |
| | acid, asparagine, or aspartic acid. |
| (ix) | FEATURE: |
| | NAME/KEY: Modified site |
| | LOCATION: 4 |
| | (D)OTHER INFORMATION: The residue in |
| | this position is either threonine, or |
| | serine. |
| (ix) | FEATURE: |
| | NAME/KEY: Modified site |
| | LOCATION: 6 |
| | (D)OTHER INFORMATION: The residue in |
| | this position is either glutamic acid, |
| | asparagine, or aspartic acid. |
| (ix) | FEATURE: |
| | NAME/KEY: Modified site |
| | LOCATION: 8 |
| | (D)OTHER INFORMATION: The residue in |
| | this position is either glutamic acid, |
| | asparagine, or aspartic acid. |
| (ix) | FEATURE: |
| | NAME/KEY: Modified site |
| | LOCATION: 9 |
| | (D) OTHER INFORMATION: The residue in |
| | this position is either threonine, or |
| | serine. |
| (ix) | FEATURE: |
| | NAME/KEY: Modified site |
| | (B) LOCATION: 11 |
| | (D) OTHER INFORMATION: The residue in |
| | this position is either threonine, or |
| | serine. |
| (ix) | FEATURE: |
| | (A) NAME/KEY: Modified site |
| | (B) LOCATION: 13 |
| | (D) OTHER INFORMATION: The residue in |
| | this position is either tyrosine, or |
| | phenylalanine. |
| (ix) | FEATURE: |
| | NAME/KEY: Modified site |
| | (B) LOCATION: 14 |
| | (D) OTHER INFORMATION: The residue in |
| | this position is either glutamic acid, |
| | asparagine, or aspartic acid. |
| (ix) | FEATURE: |
| | (A) NANE/KEY: Modified site |
| | (B) LOCATION: 15 |
| | (D) OTHER INFORMATION: The residue in |
| | this position is either threonine, or |
| | serine. |
| (vi) | ORIGINAL SOURCE: |
| | (A) ORGANISM: |
| (xi) | SEQUENCE DESCRIPTION: SEQ ID NO:88: |

```
Xaa Trp Gln Xaa Asp Xaa Cys Xaa Xaa Cys Xaa Cys Xaa Xaa Xaa
    1 5 10 15
SEQUENCE DESCRIPTION: SEQ ID NO:89:
Glu Trp Gln Thr Asp Asn Cys Glu Thr Cys Thr Cys Tyr Glu Thr Glu
    1 5 10 15
Trp Gln Thr Asp Asn Cys Glu Thr Cys Thr Cys Tyr Glu Thr
                          20 25 30
SEQUENCE DESCRIPTION: SEQ ID NO:90:
Glu Trp Gln Thr Asp Asn Cys Glu Thr Cys Thr Cys Tyr Glu Thr Glu
    1 5 10 15
Trp Gln Thr Asp Asn Cys Glu Thr Cys Thr Cys Tyr Glu Thr Glu Trp
                          20 25 30
Gln Thr Asp Asn Cys Glu Thr Cys Thr Cys Tyr Glu Thr
                  35 40 45
SEQUENCE DESCRIPTION: SEQ ID NO:91:
Glu Trp Gln Thr Asp Asn Cys Glu Thr Cys Thr Cys Tyr Glu Thr Glu
    1 5 10 15
Trp Gln Thr Asp Asn Cys Glu Thr Cys Thr Cys Tyr Glu Thr Glu Trp
                          20 25 30
Gln Thr Asp Asn Cys Glu Thr Cys Thr Cys Tyr Glu Thr Glu Trp Gln
                  35 40 45
Thr Asp Asn Cys Glu Thr Cys Thr Cys Tyr Glu Thr
          50 55 60
SEQUENCE DESCRIPTION: SEQ ID NO:92:
Glu Tyr Gln Thr Asp Asn Cys Glu Thr Cys Thr Cys Tyr Glu Thr
    1 5 10 15
SEQUENCE DESCRIPTION: SEQ ID NO:93:
Glu Trp Asn Thr Asp Asn Cys Glu Thr Cys Thr Cys Tyr Glu Thr
1 5 10 15
SEQUENCE DESCRIPTION: SEQ ID NO: 94:
Glu Trp Gln Thr Asp Gln Ser Glu Thr Cys Thr Cys Tyr Asp Thr
    1 5 10 15
SEQUENCE DESCRIPTION: SEQ ID NO:95:
Trp Gln Thr Asp Asn Cys Glu Thr Cys Thr Cys Tyr Glu Thr
    1 5 10
SEQUENCE DESCRIPTION: SEQ ID NO:96:
Gln Thr Asp Asn Cys Glu Thr Cys Thr Cys Tyr Glu Thr
    1 5 10
SEQUENCE DESCRIPTION: SEQ ID NO:97:
Glu Trp Gln Thr Asp Asn Cys Glu Thr Cys Thr Cys
    1 5 10
SEQUENCE DESCRIPTION: SEQ ID NO:98:
Glu Trp Gln Thr Asp Asn Cys Glu Thr Cys Thr Cys D-Tyr Glu Thr
1 5 10 15
SEQUENCE DESCRIPTION: SEQ ID NO:99 (scarmbled peptide):
Thr-Cys(Acm)-Glu-Asn-Cys(Acm)-Thr-Glu-Thr-Gln-Trp-Cys(Acm)-Glu-Thr-Asp-Tyr
```

1-46. (canceled)

47. A method of inhibiting cell adhesion in a mammal having a disease or condition associated with an undesired cell adhesion, the method comprising the step of administering to said mammal a compound, or a pharmaceutical composition comprising a compound selected from the group consisting of;
a) SEQ ID NO.:5,
b) a SEQ ID NO.:5 derivative, analog or fragment selected from the group consisting of SEQ ID NO.:7, any one of SEQ ID NO.:9 to SEQ ID NO.:97 and a compound having at least 80% sequence similarity with SEQ ID NO.:5, and;
c) combination of any one of a) or b) thereof.

48. The method of claim 47, wherein said compound is a SEQ ID NO.:5 derivative comprising SEQ ID NO.:5 and a grouping for increasing the stability of the compound.

49. The method of claim 48, wherein said grouping is an acetylaminomethyl moiety attached to a sulfur atom of a cysteine.

50. The method of claim 49, wherein said compound is SEQ ID NO.:7.

51. The method as defined in claim 47, wherein said adhesion is mediated though an integrin.

52. The method as defined in claim 51, wherein said integrin is CD44.

53. A method of inhibiting cell migration in a mammal having a disease or condition associated with an undesired cell migration, the method comprising the step of administering to said mammal a compound, or a pharmaceutical composition comprising a compound selected from the group consisting of;
a. SEQ ID NO.:5,
b. a SEQ ID NO.:5 derivative, analog or fragment selected from the group consisting of SEQ ID NO.:7, any one of SEQ ID NO.:9 to SEQ ID NO.:97 and a compound having at least 80% sequence similarity with SEQ ID NO.:5, and;
c. combination of any one of a) or b) thereof.

54. The method of claim 53, wherein said compound is a SEQ ID NO.:5 derivative comprising SEQ ID NO.:5 and a grouping for increasing the stability of the compound.

55. The method of claim 54, wherein said grouping is an acetylaminomethyl moiety attached to a sulfur atom of a cysteine.

56. The method of claim 55, wherein said compound is SEQ ID NO.:7.

57. A method of inhibiting cell migration or cell adhesion comprising the step of contacting cells with a compound or a pharmaceutical composition comprising a compound selected from the group consisting of;
a. SEQ ID NO.:5,
b. a SEQ ID NO.:5 derivative, analog or fragment selected from the group consisting of SEQ ID NO.:7, any one of SEQ ID NO.:9 to SEQ ID NO.:97 and a compound having at least 80% sequence similarity with SEQ ID NO.:5, and;
c. combination of any one of a) or b) thereof.

58. The method of claim 57, wherein said compound is a SEQ ID NO.:5 derivative comprising SEQ ID NO.:5 and a grouping for increasing the stability of the compound.

59. The method of claim 58, wherein said grouping is an acetylaminomethyl moiety attached to a sulfur atom of a cysteine.

60. The method of claim 59, wherein said compound is SEQ ID NO.:7.


SEQUENCE LISTING (99 sequences)

SEQ ID NO: 1 - protein, 94 aa, Homo sapiens
SCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 2 - protein, 102 aa, Artificial
  PSP94 family member
EAEAYVEFSCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 3 - protein, 10 aa, Artificial
  PSP94 family member
YTCSVSEPGI

SEQ ID NO: 4 - protein, 15 aa, Artificial
  PSP94 family member
NEGVPGDSTRKCMDL

SEQ ID NO: 5 - protein, 15 aa, Artificial
  PSP94 family member
EWQTDNCETCTCYET

SEQ ID NO: 6 - protein, 19 aa, Artificial
  PSP94 family member
IVVEKKDPKKTCSVSEWII

SEQ ID NO: 7 - protein, 15 aa, Artificial
  PCK3145 derivative
EWQTDNCETCTCYET

SEQ ID NO: 8 - DNA, 285 bp, Artificial
  cDNA
tcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatctaa

SEQ ID NO: 9 - protein, 16 aa, Artificial
  PCK3145 derivative
EWQTDNCETCTCYETE

SEQ ID NO: 10 - protein, 17 aa, Artificial
  PCK3145 derivative
EWQTDNCETCTCYETEI

SEQ ID NO: 11 - protein, 18 aa, Artificial
  PCK3145 derivative
EWQTDNCETCTCYETEIS

SEQ ID NO: 12 - protein, 19 aa, Artificial
  PCK3145 derivative
EWQTDNCETCTCYETEISC

SEQ ID NO: 13 - protein, 20 aa, Artificial
  PCK3145 derivative
EWQTDNCETCTCYETEISCC

SEQ ID NO: 14 - protein, 21 aa, Artificial
  PCK3145 derivative
EWQTDNCETCTCYETEISCCT

SEQ ID NO: 15 - protein, 22 aa, Artificial
  PCK3145 derivative
EWQTDNCETCTCYETEISCCTL

SEQ ID NO: 16 - protein, 23 aa, Artificial
  PCK3145 derivative
EWQTDNCETCTCYETEISCCTLV

SEQ ID NO: 17 - protein, 24 aa, Artificial
  PCK3145 derivative
EWQTDNCETCTCYETEISCCTLVS

SEQ ID NO: 18 - protein, 25 aa, Artificial
  PCK3145 derivative
EWQTDNCETCTCYETEISCCTLVST

SEQ ID NO: 19 - protein, 26 aa, Artificial
  PCK3145 derivative
EWQTDNCETCTCYETEISCCTLVSTP

SEQ ID NO: 20 - protein, 27 aa, Artificial
  PCK3145 derivative
EWQTDNCETCTCYETEISCCTLVSTPV

SEQ ID NO: 21 - protein, 28 aa, Artificial
  PCK3145 derivative
EWQTDNCETCTCYETEISCCTLVSTPVG

SEQ ID NO: 22 - protein, 29 aa, Artificial
  PCK3145 derivative
EWQTDNCETCTCYETEISCCTLVSTPVGY

SEQ ID NO: 23 - protein, 30 aa, Artificial
  PCK3145 derivative
EWQTDNCETCTCYETEISCCTLVSTPVGYD

SEQ ID NO: 24 - protein, 31 aa, Artificial
  PCK3145 derivative
EWQTDNCETCTCYETEISCCTLVSTPVGYDK

SEQ ID NO: 25 - protein, 32 aa, Artificial
  PCK3145 derivative
EWQTDNCETCTCYETEISCCTLVSTPVGYDKD

SEQ ID NO: 26 - protein, 33 aa, Artificial
  PCK3145 derivative
EWQTDNCETCTCYETEISCCTLVSTPVGYDKDN

SEQ ID NO: 27 - protein, 34 aa, Artificial
  PCK3145 derivative
EWQTDNCETCTCYETEISCCTLVSTPVGYDKDNC

SEQ ID NO: 28 - protein, 35 aa, Artificial
  PCK3145 derivative
EWQTDNCETCTCYETEISCCTLVSTPVGYDKDNCQ

SEQ ID NO: 29 - protein, 36 aa, Artificial
  PCK3145 derivative
EWQTDNCETCTCYETEISCCTLVSTPVGYDKDNCQR

SEQ ID NO: 30 - protein, 37 aa, Artificial
  PCK3145 derivative
EWQTDNCETCTCYETEISCCTLVSTPVGYDKDNCQRI

SEQ ID NO: 31 - protein, 38 aa, Artificial
  PCK3145 derivative
EWQTDNCETCTCYETEISCCTLVSTPVGYDKDNCQRIF

SEQ ID NO: 32 - protein, 39 aa, Artificial
  PCK3145 derivative
EWQTDNCETCTCYETEISCCTLVSTPVGYDKDNCQRIFK

SEQ ID NO: 33 - protein, 40 aa, Artificial
  PCK3145 derivative
EWQTDNCETCTCYETEISCCTLVSTPVGYDKDNCQRIFKK

SEQ ID NO: 34 - protein, 41 aa, Artificial
  PCK3145 derivative
EWQTDNCETCTCYETEISCCTLVSTPVGYDKDNCQRIFKKE

SEQ ID NO: 35 - protein, 42 aa, Artificial
  PCK3145 derivative
EWQTDNCETCTCYETEISCCTLVSTPVGYDKDNCQRIFKKED

SEQ ID NO: 36 - protein, 43 aa, Artificial
  PCK3145 derivative
EWQTDNCETCTCYETEISCCTLVSTPVGYDKDNCQRIFKKEDC

SEQ ID NO: 37 - protein, 44 aa, Artificial
  PCK3145 derivative
EWQTDNCETCTCYETEISCCTLVSTPVGYDKDNCQRIFKKEDCK

SEQ ID NO: 38 - protein, 45 aa, Artificial
  PCK3145 derivative
EWQTDNCETCTCYETEISCCTLVSTPVGYDKDNCQRIFKKEDCKY

SEQ ID NO: 39 - protein, 46 aa, Artificial
  PCK3145 derivative
EWQTDNCETCTCYETEISCCTLVSTPVGYDKDNCQRIFKKEDCKYI

SEQ ID NO: 40 - protein, 47 aa, Artificial
  PCK3145 derivative
EWQTDNCETCTCYETEISCCTLVSTPVGYDKDNCQRIFKKEDCKYIV

SEQ ID NO: 41 - protein, 48 aa, Artificial
  PCK3145 derivative
EWQTDNCETCTCYETEISCCTLVSTPVGYDKDNCQRIFKKEDCKYIVV

SEQ ID NO: 42 - protein, 49 aa, Artificial
  PCK3145 derivative
EWQTDNCETCTCYETEISCCTLVSTPVGYDKDNCQRIFKKEDCKYIVVE

SEQ ID NO: 43 - protein, 50 aa, Artificial
  PCK3145 derivative
EWQTDNCETCTCYETEISCCTLVSTPVGYDKDNCQRIFKKEDCKYIVVEK

SEQ ID NO: 44 - protein, 51 aa, Artificial
  PCK3145 derivative
EWQTDNCETCTCYETEISCCTLVSTPVGYDKDNCQRIFKKEDCKYIVVEKK

SEQ ID NO: 45 - protein, 52 aa, Artificial
  PCK3145 derivative
EWQTDNCETCTCYETEISCCTLVSTPVGYDKDNCQRIFKKEDCKYIVVEKKD

SEQ ID NO: 46 - protein, 53 aa, Artificial
  PCK3145 derivative
EWQTDNCETCTCYETEISCCTLVSTPVGYDKDNCQRIFKKEDCKYIVVEKKDP

SEQ ID NO: 47 - protein, 54 aa, Artificial
  PCK3145 derivative
EWQTDNCETCTCYETEISCCTLVSTPVGYDKDNCQRIFKKEDCKYIVVEKKDPK

SEQ ID NO: 48 - protein, 55 aa, Artificial
  PCK3145 derivative
EWQTDNCETCTCYETEISCCTLVSTPVGYDKDNCQRIFKKEDCKYIVVEKKDPKK

SEQ ID NO: 49 - protein, 56 aa, Artificial
  PCK3145 derivative
EWQTDNCETCTCYETEISCCTLVSTPVGYDKDNCQRIFKKEDCKYIVVEKKDPKKT

SEQ ID NO: 50 - protein, 57 aa, Artificial
  PCK3145 derivative
EWQTDNCETCTCYETEISCCTLVSTPVGYDKDNCQRIFKKEDCKYIVVEKKDPKKTC

SEQ ID NO: 51 - protein, 58 aa, Artificial
  PCK3145 derivative
EWQTDNCETCTCYETEISCCTLVSTPVGYDKDNCQRIFKKEDCKYIVVEKKDPKKTCS

SEQ ID NO: 52 - protein, 59 aa, Artificial
  PCK3145 derivative
EWQTDNCETCTCYETEISCCTLVSTPVGYDKDNCQRIFKKEDCKYIVVEKKDPKKTCSV

SEQ ID NO: 53 - protein, 60 aa, Artificial
  PCK3145 derivative
EWQTDNCETCTCYETEISCCTLVSTPVGYDKDNCQRIFKKEDCKYIVVEKKDPKKTCSVS

SEQ ID NO: 54 - protein, 61 aa, Artificial
  PCK3145 derivative
EWQTDNCETCTCYETEISCCTLVSTPVGYDKDNCQRIFKKEDCKYIVVEKKDPKKTCSVSE

SEQ ID NO: 55 - protein, 62 aa, Artificial
  PCK3145 derivative
EWQTDNCETCTCYETEISCCTLVSTPVGYDKDNCQRIFKKEDCKYIVVEKKDPKKTCSVSEW

SEQ ID NO: 56 - protein, 63 aa, Artificial
  PCK3145 derivative
EWQTDNCETCTCYETEISCCTLVSTPVGYDKDNCQRIFKKEDCKYIVVEKKDPKKTCSVSEWI

SEQ ID NO: 57 - protein, 64 aa, Artificial
  PCK3145 derivative
EWQTDNCETCTCYETEISCCTLVSTPVGYDKDNCQRIFKKEDCKYIVVEKKDPKKTCSVSEWII

SEQ ID NO: 58 - protein, 16 aa, Artificial
  PCK3145 derivative
SEWQTDNCETCTCYET

SEQ ID NO: 59 - protein, 17 aa, Artificial
  PCK3145 derivative
NSEWQTDNCETCTCYET

SEQ ID NO: 60 - protein, 18 aa, Artificial
  PCK3145 derivative
INSEWQTDNCETCTCYET

SEQ ID NO: 61 - protein, 19 aa, Artificial
  PCK3145 derivative
PINSEWQTDNCETCTCYET

SEQ ID NO: 62 - protein, 20 aa, Artificial
  PCK3145 derivative
HPINSEWQTDNCETCTCYET

SEQ ID NO: 63 - protein, 21 aa, Artificial
  PCK3145 derivative
KHPINSEWQTDNCETCTCYET

SEQ ID NO: 64 - protein, 22 aa, Artificial
  PCK3145 derivative
NKHPINSEWQTDNCETCTCYET

SEQ ID NO: 65 - protein, 23 aa, Artificial
  PCK3145 derivative
GNKHPINSEWQTDNCETCTCYET

SEQ ID NO: 66 - protein, 24 aa, Artificial
  PCK3145 derivative
KGNKHPINSEWQTDNCETCTCYET

SEQ ID NO: 67 - protein, 25 aa, Artificial
  PCK3145 derivative
LKGNKHPINSEWQTDNCETCTCYET

SEQ ID NO: 68 - protein, 26 aa, Artificial
  PCK3145 derivative
DLKGNKHPINSEWQTDNCETCTCYET

SEQ ID NO: 69 - protein, 27 aa, Artificial
  PCK3145 derivative
MDLKGNKHPINSEWQTDNCETCTCYET

SEQ ID NO: 70 - protein, 28 aa, Artificial
  PCK3145 derivative
CMDLKGNKHPINSEWQTDNCETCTCYET

SEQ ID NO: 71 - protein, 29 aa, Artificial
  PCK3145 derivative
KCMDLKGNKHPINSEWQTDNCETCTCYET

SEQ ID NO: 72 - protein, 30 aa, Artificial
  PCK3145 derivative
RKCMDLKGNKHPINSEWQTDNCETCTCYET

SEQ ID NO: 73 - protein, 31 aa, Artificial
  PCK3145 derivative
TRKCMDLKGNKHPINSEWQTDNCETCTCYET

SEQ ID NO: 74 - protein, 32 aa, Artificial
  PCK3145 derivative
STRKCMDLKGNKHPINSEWQTDNCETCTCYET

SEQ ID NO: 75 - protein, 33 aa, Artificial
  PCK3145 derivative
DSTRKCMDLKGNKHPINSEWQTDNCETCTCYET

SEQ ID NO: 76 - protein, 34 aa, Artificial
  PCK3145 derivative
GDSTRKCMDLKGNKHPINSEWQTDNCETCTCYET

SEQ ID NO: 77 - protein, 35 aa, Artificial
  PCK3145 derivative
PGDSTRKCMDLKGNKHPINSEWQTDNCETCTCYET

SEQ ID NO: 78 - protein, 36 aa, Artificial
  PCK3145 derivative
VPGDSTRKCMDLKGNKHPINSEWQTDNCETCTCYET

SEQ ID NO: 79 - protein, 37 aa, Artificial
  PCK3145 derivative
GVPGDSTRKCMDLKGNKHPINSEWQTDNCETCTCYET

SEQ ID NO: 80 - protein, 38 aa, Artificial
  PCK3145 derivative
EGVPGDSTRKCMDLKGNKHPINSEWQTDNCETCTCYET

SEQ ID NO: 81 - protein, 39 aa, Artificial
  PCK3145 derivative
NEGVPGDSTRKCMDLKGNKHPINSEWQTDNCETCTCYET

SEQ ID NO: 82 - protein, 40 aa, Artificial
  PCK3145 derivative
PNEGVPGDSTRKCMDLKGNKHPINSEWQTDNCETCTCYET

SEQ ID NO: 83 - protein, 41 aa, Artificial
  PCK3145 derivative
IPNEGVPGDSTRKCMDLKGNKHPINSEWQTDNCETCTCYET

SEQ ID NO: 84 - protein, 42 aa, Artificial
  PCK3145 derivative
FIPNEGVPGDSTRKCMDLKGNKHPINSEWQTDNCETCTCYET

SEQ ID NO: 85 - protein, 43 aa, Artificial
  PCK3145 derivative
YFIPNEGVPGDSTRKCMDLKGNKHPINSEWQTDNCETCTCYET

SEQ ID NO: 86 - protein, 44 aa, Artificial
  PCK3145 derivative
CYFIPNEGVPGDSTRKCMDLKGNKHPINSEWQTDNCETCTCYET

SEQ ID NO: 87 - protein, 45 aa, Artificial
  PCK3145 derivative
SCYFIPNEGVPGDSTRKCMDLKGNKHPINSEWQTDNCETCTCYET

SEQ ID NO: 88 - protein, 15 aa, Artificial
  PCK3145 analog
XWQXDXCXXCXCXXX

SEQ ID NO: 89 - protein, 30 aa, Artificial
  PCK3145 derivative
EWQTDNCETCTCYETEWQTDNCETCTCYET

SEQ ID NO: 90 - protein, 45 aa, Artificial
  PCK3145 derivative
EWQTDNCETCTCYETEWQTDNCETCTCYETEWQTDNCETCTCYET

SEQ ID NO: 91 - protein, 60 aa, Artificial
  PCK3145 derivative
EWQTDNCETCTCYETEWQTDNCETCTCYETEWQTDNCETCTCYETEWQTDNCETCTCYET

SEQ ID NO: 92 - protein, 15 aa, Artificial
  PCK3145 analog
EYQTDNCETCTCYET

SEQ ID NO: 93 - protein, 15 aa, Artificial
  PCK3145 analog
EWNTDNCETCTCYET

SEQ ID NO: 94 - protein, 15 aa, Artificial
  PCK3145 analog
EWQTDQSETCTCYDT

SEQ ID NO: 95 - protein, 14 aa, Artificial
  PCK3145 fragment
WQTDNCETCTCYET

SEQ ID NO: 96 - protein, 13 aa, Artificial
  PCK3145 fragment
QTDNCETCTCYET

SEQ ID NO: 97 - protein, 12 aa, Artificial
  PCK3145 fragment
EWQTDNCETCTC

SEQ ID NO: 98 - protein, 15 aa, Artificial
  PCK3145 derivative
EWQTDNCETCTCXET

SEQ ID NO: 99 - protein, 15 aa, Artificial
  scrambled peptide
TCENCTETQWCETDY